(19) United States
(12) Patent Application Publication
(10) Pub. No.: US 2023/0407398 A1
(43) Pub. Date: Dec. 21, 2023

(54) Methods and Systems for Detecting Methylation of Fragile X

(71) Applicant: Laboratory Corporation of America Holdings, Burlington, NC (US)

(72) Inventors: Patricia Okamoto, Shrewsbury, MA (US); Zhenxi Zhang, Westborough, MA (US)

(21) Appl. No.: 18/107,877

(22) Filed: Feb. 9, 2023

Related U.S. Application Data
(60) Provisional application No. 63/308,242, filed on Feb. 9, 2022.

(51) Int. Cl. C12Q 1/6883

(52) U.S. Cl. CPC C12Q 1/6883; C12Q 2600/154

(57) ABSTRACT

Disclosed are methods and systems for detecting methylation of the Fragile X FMRI gene. In certain embodiments, the DNA is fragmented and then the assay uses methylation-specific immunoprecipitation to separate the genomic DNA into a methylated and an unmethylated fraction. In an embodiment, both fractions, along with the initial unfractionated DNA, are processed in parallel. In certain embodiments, the assay in performed on a plurality of samples using a multiwell plate. After resolution of the PCR products by capillary electrophoresis, a computerized custom calling tool may be used to qualitatively determine methylation status.

### RELATED APPLICATIONS

This application claims priority to U.S. Provisional Application No. 63/308,242, filed Feb. 9, 2022. The disclosure of U.S. Provisional Application No. 63/308,242 is incorporated by reference in its entirety herein.

### SEQUENCE LISTING

The instant application contains a Sequence Listing which has been submitted electronically in xml format and is hereby incorporated by reference in its entirety. The file with the sequence listing, created on Feb. 7, 2023, is named LC 2022-04-US (057618-1360256).xml and is 4,703 bytes in size.

### FIELD

Disclosed are methods and systems for detecting methylation of the Fragile X FMR1 gene.

### BACKGROUND

Fragile X syndrome (FXS), formerly known as Martin-Bell syndrome, is the second most common cause of inherited mental disability (behind Down syndrome), affecting approximately 1 in 4000 males and 1 in 8000 females. “Fragile X” derives its name from the cytogenetic fragile site at Xq27.3 that appears when cells of affected individuals are cultured in folate-deficient medium. Affected males exhibit developmental delay and intellectual disability, mild dysmorphic features, macroorchidism, and high-pitched jocular speech. Females usually show less severe phenotype, which is typical for X chromosome-linked diseases. As expected with a neurodevelopmental disorder, IQ tends to deteriorate with age. Many patients also exhibit subtle connective tissue abnormalities, hyperactive attention deficit disorder and autistic-like behavior. The phenotype is associated with mutations in the FMRI gene located on Xq27.3 (reviewed in Hagerman, R., et al., Fragile X syndrome, Nat Rev Dis Primers 2017, 3:17065).

FMRI is a highly conserved gene that consists of 17 exons and spans ˜38 Kb. Within the 4.4 Kb of the FMRI transcript, is a CGG trinucleotide repeat region located in the 5′-untranslated region (UTR). The gene encodes an RNA-binding protein that regulates protein translation of many genes involved in nervous system synaptic functions. FXS is caused by FMRI protein (FMRP) deficiency. For >98% of cases, loss of FMRP results from hypermethylation of the FMRI 5′UTR region, which is triggered by CGG repeat expansion (reviewed in Hagerman et al., 2017). However, there are challenges with current assay systems, such as Southern blot detection of FMRI methylation status due to for example, processing time and the amount of input DNA required. Thus, there is a need for improved methods and systems to measure alterations of the FMRI gene and methylation patterns associated with such alterations that can lead to FMRP deficiency. In particular, there is a need for methods and systems that can increase the accuracy and specificity of such assays.

### SUMMARY

Disclosed are methods and systems for detecting methylation of the fragile X FMRI gene. The methods and systems may be embodied in a variety of ways.

In certain embodiments, disclosed is a method for determining methylation of an FMRI gene in a sample from a subject comprising the steps of: isolating total nucleic acid from the sample, the total nucleic acid comprising both methylated nucleic acid and unmethylated nucleic acid; contacting a first portion of the isolated total nucleic acid with a methyl binding protein; isolating a portion of the unbound nucleic acid, thereby generating a nucleic acid fraction enriched for unmethylated nucleic acid; isolating the bound nucleic acid, thereby generating a nucleic acid fraction enriched for methylated nucleic acid; conducting PCR amplification of the FMRI gene using an aliquot of nucleic acid from the fraction enriched for methylated nucleic acid; conducting PCR amplification of the FMRI gene using an aliquot of nucleic acid from the fraction enriched for unmethylated nucleic acid; and determining the presence or absence of an FMRI amplification product in both the fraction enriched for methylated nucleic acid and the fraction enriched for unmethylated nucleic acid.

Also disclosed are compositions, kits, and systems for performing the disclosed methods or any of the steps of the disclosed methods.

### BRIEF DESCRIPTION OF THE DRAWINGS

The disclosure may be better understood by reference to the following non-limiting drawings.

FIG. 1 shows a method for determining Fragile X (FMRI) methylation status according to an embodiment of the disclosure.

FIG. 2 shows results of sonication of DNA isolated from various sample types according to an embodiment of the disclosure.

FIG. 3 shows typical sheared DNA size distribution for 24 fresh blood DNA samples according to an embodiment of the disclosure. The x axis shows size (bp) and the y axis shows relative fluorescence units (rfu). The peaks labeled 1 and 20,000 are internal sizing standards indicating the range of the analyzer.

FIG. 4 shows a format for binding methylated DNA to a methyl CpG binding domain (MBD)-containing protein MBD2a according to an embodiment of the disclosure.

FIG. 5 shows a workflow scheme for an analysis of the FMRI gene according to an embodiment of the disclosure.

FIG. 6 shows a method for determining the size of FMRI alleles that are either methylated, unmethylated or partially method according to an embodiment of the disclosure.

FIG. 7 shows a system for determining Fragile X (FMRI) methylation status according to an embodiment of the disclosure.

FIG. 8 shows an exemplary computing device according to various embodiments of the disclosure.

FIG. 9 shows an evaluation of the methylation status of an expanded 75 bp CGG repeat in a sample (AFC4) as a fully methylated allele in accordance with an embodiment of the disclosure. The y axis indicates rfu and the average size of various CGG repeat (rpt) regions are shown. Total indicates total DNA; Meth indicates DNA enriched for methylated DNA, and Unmeth indicates DNA enriched for unmethylated DNA.

FIG. 10 shows an evaluation of the methylation status of an expanded 163 bp CGG repeat in a sample (BLC2) as a partially methylated allele (i.e., detected in both methylated and unmethylated DNA) in accordance with an embodiment of the disclosure. The y axis indicates rfu and the average size of various CGG repeat (rpt) regions are shown. Total indicates total DNA; Meth indicates DNA enriched for methylated DNA, and Unmeth indicates DNA enriched for unmethylated DNA.

FIG. 11 shows an evaluation of the methylation status of an unstable expanded 162 bp CGG repeat allele and a FM allele in a sample (BL7) as unmethylated in accordance with an embodiment of the disclosure. The y axis indicates rfu and the average size of various CGG repeat (rpt) regions are shown. Total indicates total DNA; Meth indicates DNA enriched for methylated DNA, and Unmeth indicates DNA enriched for unmethylated DNA.

### DETAILED DESCRIPTION

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

### Definitions

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

As used herein, the term “detectable moiety” or “detectable biomolecule” or “reporter” refers to a molecule that can be measured in a quantitative assay. For example, a detectable moiety may comprise an enzyme that may be used to convert a substrate to a product that can be measured (e.g., a visible product). Or, a detectable moiety may be a radioisotope that can be quantified. Or, a detectable moiety may be a fluorophore. Or, a detectable moiety may be a luminescent molecule. Or, other detectable molecules may be used.

The terms “labeled” and “labeled with a detectable agent or moiety” are used herein interchangeably to specify that an entity (e.g., a nucleic acid probe, antibody) can be measured by detection of the label (e.g., visualized, detection of radioactivity, fluorescence and the like) for example following binding to another entity (e.g., a nucleic acid, polypeptide). The detectable agent or moiety may be selected such that it generates a signal which can be measured and whose intensity is related to (e.g., proportional to) the amount of bound entity. A wide variety of systems for labeling and/or detecting nucleic acids are known in the art. Labeled nucleic acids can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes, colorimetric labels, magnetic labels, haptens, molecular beacons, aptamer beacons, and the like.

As used herein, the term “biological sample” or “sample” refers to a sample obtained from a biological source, including, but not limited to, an animal, a cell culture, an organ culture, and the like. Suitable samples include whole blood, amniotic fluid, amniotic fluid cell cultures, chorionic villus sampling, chorionicvillus sample cell cultures, saliva and buccals, as well as cell-free DNA, plasma, serum, urine, tear, cerebrospinal fluid, organ, hair, muscle, or other tissue samples. In an embodiment, the sample comprises prenatal nucleic acid.

As used herein, a “subject” may comprise an animal. Thus, in some embodiments, the biological sample is obtained from a mammalian animal, including, but not limited to a human or fetus, a dog, a cat, a horse, a rat, a monkey, and the like. In some embodiments, the biological sample is obtained from a human subject. In some cases, the human subject is a pregnant female. In some embodiments the subject is prenatal (i.e., fetal DNA sample from a pregnant female). In some embodiments, the subject is a patient, that is, a living person presenting themselves in a clinical setting for diagnosis, prognosis, or treatment of a disease or condition

Total nucleic acid as used herein is nucleic acid isolated from a sample that has not been subjected for selection of methylated nucleic acid sequences.

A nucleic acid fraction enriched for methylated nucleic acid is a nucleic acid sample that has been processed to enrich for nucleic acid sequences that are methylated as compared to nucleic acid sequences that are not methylated.

A nucleic acid fraction enriched for unmethylated nucleic acid is a nucleic acid sample that has been processed to enrich for nucleic acid sequences that are unmethylated as compared to nucleic acid sequences that are methylated.

PCR amplification of the FMRI gene as used herein refers to polymerase chain reaction (PCR) amplification of at least a portion of the FMRI gene using primers having sequences specific for at least a portion of the FMRI gene, or primers that have sequences specific for sequences that are upstream (i.e., 5′) and/or downstream (i.e., 3′) of the FMRI gene so as to amplify the entire gene and the additional upstream or downstream sequences contained in the primer sequences.

FRAX mePCR assay as used herein is a PCR assay used to determine if FMRI gene sequences are present in a nucleic acid fraction enriched for methylated nucleic acid or a nucleic acid fraction enriched for unmethylated nucleic acid or both. In some aspects, FRAX PCR employs GS-PCR.

FRAX PCR assay as used herein is a PCR assay used to determine the size of an FMRI gene alleles in a subject. In some aspects, FRAX PCR employs GS-PCR. The assay optionally includes a determination of the number of expanded CGG repeats and a determination of the sex of the subject from whom the sample was obtained.

GS-PCR as used herein refers to gene-specific (GS) PCR of the FMRI gene. GS-PCR may employ primers that are positioned upstream (i.e., 5′) and downstream (i.e., 3′) of the FMRI promoter region containing CGG repeats.

An expanded FMRI allele refers to an allele of the FMRI gene that has an expanded CGG triplet repeat region as compared to a normal control. For example, an expanded allele may be classified as a premutation (PM) of about 55-200 CGG repeats, and/or full mutation (FM) of >200 CGG repeats alleles. Generally, the size of the CGG repeat region <about 55 CGG repeats may be classified as normal.

### FMRI Methylation Assay

Disclosed are methods for detecting methylation of the FMRI gene. The methods may be embodied in a variety of ways.

In certain embodiments, disclosed is a fragile X (FRAX) methylation PCR (mePCR) assay method, and systems for performing the method, that overcome many of the testing limitations currently encountered in Southern blot (SB) analysis for methylation detection. In certain embodiments, the assay uses methylation-specific immunoprecipitation to separate the genomic DNA into a methylated and an unmethylated fraction. In an embodiment, both fractions, along with the initial unfractionated DNA, are processed in parallel. In certain embodiments, the assay in performed on a plurality of samples using a multiwell plate. After resolution of the PCR products by capillary electrophoresis or other size selection method, a computerized custom calling method as disclosed herein may be used to qualitatively determine methylation status. In addition to whole blood and prenatal specimens, saliva and buccal specimens have been validated to expand the utility of the FRAX mePCR assay.

Thus, in certain embodiments, disclosed is a method for determining methylation of an FMRI gene in a sample from a subject comprising the steps of: isolating total nucleic acid from the sample, the total nucleic acid comprising both methylated nucleic acid and unmethylated nucleic acid; processing the nucleic acid to partially purify unmethylated nucleic acid and methylated nucleic acid thereby generating a nucleic acid fraction enriched for unmethylated nucleic acid and a nucleic acid fraction enriched for methylated nucleic acid; conducting PCR amplification of the FMRI gene using an aliquot of nucleic acid from the fraction enriched for methylated nucleic acid; conducting PCR amplification of the FMRI gene using an aliquot of nucleic acid from the fraction enriched for unmethylated nucleic acid; and determining the presence or absence of an FMRI amplification product in both the fraction enriched for methylated nucleic acid and the fraction enriched for unmethylated nucleic acid.

For example, disclosed is a method for determining methylation of an FMRI gene in a sample from a subject comprising the steps of: isolating total nucleic acid from the sample, the total nucleic acid comprising both methylated nucleic acid and unmethylated nucleic acid; contacting a first portion of the isolated total nucleic acid with a methyl binding protein; isolating a portion of the unbound nucleic acid, thereby generating a nucleic acid fraction enriched for unmethylated nucleic acid; isolating the bound nucleic acid, thereby generating a nucleic acid fraction enriched for methylated nucleic acid; conducting PCR amplification of the FMRI gene using an aliquot of nucleic acid from the fraction enriched for methylated nucleic acid; conducting PCR amplification of the FMRI gene using an aliquot of nucleic acid from the fraction enriched for unmethylated nucleic acid; and determining the presence or absence of an FMRI amplification product in both the fraction enriched for methylated nucleic acid and the fraction enriched for unmethylated nucleic acid.

As noted herein, the subject may be a human, as for example a pregnant female. Or, the subject may be a fetus carried by a pregnant female. Suitable samples may include whole blood, amniotic fluid, amniotic fluid cell cultures, chorionic villus sampling, chorionicvillus sample cell cultures, saliva and buccals, as well as cell-free DNA, plasma, serum, urine, tear, cerebrospinal fluid, organ, hair, muscle, or other tissue samples. In an embodiment, the sample comprises prenatal nucleic acid.

In certain embodiments, the method may further comprise determining the size of the FMRI amplification product in at least the one of the fraction enriched for methylated nucleic acid and/or the fraction enriched for unmethylated nucleic acid. In certain embodiments, the size of the amplification product is determined using primers that are positioned upstream (i.e., 5′) and downstream (i.e., 3′) of the FMRI gene (i.e., GS-PCR). Thus, in an embodiment, the size of the FMRI gene (and promoter region) in either the methylated nucleic acid fraction or the unmethylated fraction may be determined. In an embodiment, the method may further comprise determining the number of CGG repeats at the FMRI gene for the amplification product in at least one of the fraction enriched for methylated nucleic acid and the fraction enriched for unmethylated nucleic acid. The number of CGG repeats may be determined based on the size of the GS-PCR product(s) using capillary electrophoresis or equivalent methods, as for example, by comparing to the number of CGG repeats in a reference genomic sequence. Thus, the disclosed methods may comprise determining whether the subject has either: (i) partial methylation of FMRI; (ii) both a methylated and unmethylated copy of FMRI, or (iii) full methylation of FMRI. Additionally and/or alternatively, the method may comprise determining whether the subject has either: (i) partial methylation of an expanded FMRI allele; (ii) both a methylated and unmethylated expanded FMRI allele; or (iii) full methylation of an expanded FMRI allele. Also, in certain embodiments, the method may comprise comparing the size of the FMRI gene in the methylated nucleic acid fraction or the unmethylated fraction to the size of the FMRI gene as detected in total nucleic acid from the subject. In such embodiments, the method may comprise PCR amplification of the FMRI gene using an aliquot of nucleic acid from the total nucleic acid and determining the size of at least one FMRI amplification product in the total nucleic acid. In certain embodiments, the method may comprise determining the number of CGG repeats at the FMRI gene for the at least one amplification product obtained from the total nucleic acid fraction. In certain embodiments, determining the size of the FMRI amplification product in at least one of the fraction enriched for methylated nucleic acid and/or the fraction enriched for unmethylated nucleic acid comprises comparing the size of the amplification products for the fraction enriched for methylated nucleic acid and the fraction enriched for unmethylated nucleic acid to the size of the at least one FMRI amplification product for total nucleic acid. Additionally and/or alternatively, the method may comprise an assessment that the size(s) of the FMRI amplification product(s) in the fraction enriched for methylated nucleic acid and the fraction enriched for unmethylated nucleic acid are the size of the at least one FMRI amplification product for total nucleic acid.

In certain embodiments, at least one primer used to generate an FMRI amplification product is labeled with a detectable moiety. For example, in certain embodiments, the at least one primer used to generate an amplification product from the FMRI gene from the methylated enriched fraction and/or the unmethylated enriched fraction and/or the total nucleic acid fraction have the same sequence but are labeled with a different detectable moiety.

Any of a wide variety of detectable agents can be used in the practice of the disclosure. Suitable detectable agents include, but are not limited to: various ligands, radionucleotides; fluorescent dyes; chemiluminescent agents (such as, for example, acridinum esters, stabilized dioxetanes, and the like); bioluminescent agents; spectrally resolvable inorganic fluorescent semiconductor nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

Below are described some non-limiting examples of some detectable moieties that may be used.

Fluorescent Dyes

In certain embodiments, a detectable moiety is a fluorescent dye. Numerous known fluorescent dyes of a wide variety of chemical structures and physical characteristics are suitable for use in the practice of the disclosure. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Q-DOTS, Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, CY-5, CY-3.5, CY5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, OR. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92:4347. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described, for example, in U.S. Pat. No. 5,945,526. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (i.e., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201T1, 212Bi, 213Bi, 211At, 153Sm, 177Lu).

Dendrimers

In some embodiments, signal amplification is achieved using labeled dendrimers as the detectable moiety (see, e.g., Physiol Genomics 3:93-99, 2000). Fluorescently labeled dendrimers are available from Genisphere (Montvale, N.J.). These may be chemically conjugated to the oligonucleotide primers by methods known in the art.

In some embodiments, the detectable moieties are dyes, such as fluorescent dyes. In certain embodiments, the primers used to amplify the FMRI gene in methylated nucleic acid have the same sequence, but one of the primers (e.g., the reverse primer) may be labeled with a different dye. Thus, in certain embodiments, the fluorescent dye used for detection of the amplification products from the fraction enriched for unmethylated nucleic acid and/or the total nucleic acid is 6-carboxyfluorescein (FAM) and the dye used for detection of the amplification product from the fraction enriched for methylated nucleic acid is hexachlorofluorescein (HEX).

Thus, in certain embodiments, the primers used to amplify the FMRI gene in unmethylated nucleic acid comprise a forward primer, FRAX-F1 (SEQ ID NO: 1) having a nucleic acid sequence of: 5′-GCT CAG CTC CGT TTC GGT TTC ACT TCC GGT-3′ and a reverse primer having the sequence of SEQ ID NO: 4 (5′-AGC CCC GCA CTT CCA CCA CCA GCT CCT CCA-3′). In an embodiment, the reverse primer is labeled at the 5′ end with the dye FAM or the dye HEX, or a different detectable moiety. Additionally, or alternatively, the forward primer (SEQ ID NO: 1) may be labeled with FAM or HEX (or a different detectable moiety).

Thus, in an embodiment the reverse primer used to amplify the FMRI gene in unmethylated nucleic acid is a forward primer FRAX-F1 (SEQ ID NO. 1) and FRAX-R-6FAM (SEQ ID NO: 2) 5′-FAM-AGC CCC GCA CTT CCA CCA CCA GCT CCT CCA-3′ (Table 1). Also, in certain embodiments, the primers used to amplify the FMRI gene in methylated nucleic acid comprise a forward primer FRAX-F1 (SEQ ID NO: 1), but the reverse primer is labeled with HEX. Thus in an embodiment the reverse primer used to amplify the FMRI gene in methylated nucleic acid is FRAX-R-HEX (SEQ ID NO: 3) 5′-HEX-AGC CCC GCA CTT CCA CCA CCA GCT CCT CCA-3′ (Table 2). Or, other combinations of the primers of SEQ ID NO: 1 and 4, labeled with different moieties may be chosen.

TABLE 1
GS-PCR primers for unmethylated DNA PCR:

| SEQ ID | Primer | |
|---|---|---|
| NO: | Name | Sequence (5′-3′) |
| 1 | FRAX-F1 | 5′-GCT CAG CTC CGT TTC |
| | | GGT TTC ACT TCC GGT-3′ |
| 2 | FRAX-R- | 5′-FAM-AGC CCC GCA CTT |
| | 6FAM | CCA CCA CCA GCT CCT CCA-3′ |

TABLE 2
GS-PCR primers for methylated DNA PCR:

| SEQ ID | Primer | |
|---|---|---|
| NO: | Name | Sequence (5′-3′) |
| 1 | FRAX-F1 | 5′-GCT CAG CTC CGT TTC |
| | | GGT TTC ACT TCC GGT-3′ |
| 3 | FRAX-R- | 5′-HEX-AGC CCC GCA CTT |
| | HEX | CCA CCA CCA GCT CCT CCA-3′ |

In certain embodiments, to optimize binding of the nucleic acid to the methyl binding protein, the nucleic acid is fragmented to fragments having a size ranging from about 2.4-3.2 kilobase pairs (kb) or about 2.5 kb or about 3 kb sized fragments prior to contacting the nucleic acid with the methyl binding protein. Or other sized fragments may be used as disclosed herein. Embodiments of generating nucleic acid fragments are disclosed in more detail herein.

In certain embodiments, the method is performed after an initial assessment of whether the sample comprises at least one FMRI allele that appears to exhibit expansion of the 5′ upstream region, as for example by expansion of a CGG trinucleotide repeat region located in the untranslated region (UTR) of the gene. Thus, the method may comprise performing a PCR assay to determine the number and/or positioning of CGG repeats at the FMRI gene in the nucleic acid from the sample. For example, in certain embodiments, the method comprises PCR amplification of the FMRI gene using an aliquot of nucleic acid from the total nucleic acid fraction and determining the size of the FMRI amplification products (FRAX PCR). If it appears that additional assessment is warranted (e.g., the sample comprises a PM or FM) the sample my be further assayed by FRAX me-PCR.

In an embodiment, the total nucleic acid may be fragmented for use in the FRAX me-PCR assay. For example, in certain embodiments the total nucleic acid may be fragmented by sonication, enzymatic digestion or other similar methods used to fragment nucleic acids. The method may further comprise confirming the number of CGG repeats at the FMRI gene for an amplification product obtained from the total nucleic acid fraction. In certain embodiments, the method may include a step to determine the size and abundance of each allele peak using GS-PCR (i.e., GS-PCR). The method may also include calculating an average CGG repeat number for each allele from the GS-PCR data and then using the size of those calculated peaks as the peaks of interest that are measured by FRAX me-PCR, i.e., to determine whether the expanded alleles are methylated or unmethylated. In certain embodiments, the analysis does not include peaks from the FRAX me-PCR that are not found in the FRAX PCR from total nucleic acid. As noted herein, in certain embodiments, a determination of the approximate size of the FMRI gene may comprise amplification with primers located 5′ and 3′ of the FMRI gene and upstream region (e.g., GS-PCR). In an embodiment, primers having the sequence SEQ ID NO: 1 for the forward primer and SEQ ID NO: 2 for the reverse primer are used for the GS-PCR (i.e., FRAX PCR) from total DNA.

Also, in certain embodiments, mosaicism in the sample is identified using FRAX PCR. For example, mosaicism may be determined by identifying the number distinctive peaks from FRAX PCR. As each peak represents an allele, it is expected to detect 1 or 2 alleles in male or female samples, respectively. Mosaicism may be identified when more than 1 or 2 alleles are detected in male or female samples, respectively.

In certain embodiments, the intended clinical use for the FRAX mePCR assay is to determine methylation status of a premutation (PM) of about 55-200 CGG repeats, and/or full mutation (FM) of >200 CGG repeats alleles. This can be important as methylation status can influence clinical severity for both PM carriers as well as FM carriers. Thus, although an unmethylated FM allele can still produce FMRI protein, it is rare to find FM allele carriers that are unmethylated. In an embodiment, the method is intended for fragile X carrier screening and diagnostic testing including at-risk prenatal specimens.

Thus, in certain embodiments, prior to determining methylation status at the FMRI locus, PCR amplification is used to determine the extent of CGG expansion and the sex of the individual for whom the FMRI status is being assessed. Thus, in some embodiments, prior to testing in the FRAX mePCR assay, a determination of whether the subject may comprise either no mutation (i.e., no expansion, a premutation i.e., 55-200 CGG repeats) or a full mutation (i.e., >200 CGG repeats) as well as a determination of the sex is performed. The determination of the nature of CGG expansion and/or sex may, in certain embodiments, comprise three parts: i) gene-specific PCR (GS-PCR) to determine the total number of repeats on each allele; (ii) optionally, triplet-primed PCR (TRP-PCR) to screen for repeat expansions; and (iii) gender-detection PCR. In an embodiment, the initial GS-PCR may use labeled primers to facilitate analysis (e.g., using instrumentation used for Sanger sequencing). Or, unlabeled primers may be used for detection e.g., using a fragment analyzer, bioanalyzer and the like. If the sample is determined as having either a premutation (PM) or a full mutation (FM) an assessment of the methylation status for each FMRI allele may be performed.

In certain embodiments, the methyl binding protein is a bifunctional polypeptide comprising: (i) an Fc portion of an antibody; (ii) a short flexible peptide linker; and (iii) a DNA-binding domain of an MBD2 protein. In certain embodiments, the methyl binding protein is provided as the EpiMark® Methylated DNA Enrichment Kit, from New England Biolabs (Ipswich, MA). The EpiMark Kit provides a methyl-CpG binding domain of human MBD2 protein fused to the Fc-tail of human IgG1 (MBD2-Fc). Or other methyl binding proteins, such as the Active Motifs MethylCollector™ Ultra, available from Active Motif, Inc. (Carlsbad, CA) may be used. Or, other methyl-binding proteins may be used.

In certain embodiments, the amplification products may be analyzed by capillary electrophoresis. Or other sizing techniques, such as for example, gel electrophoresis or melting curve analysis (i.e., the size of PCR products may be determined by their melting temperatures) may be used. The results can be used to determine for each of the alleles identified in total nucleic acid whether the subject has no methylation, partial methylation of FMK/(having both a methylated and unmethylated copy of FMRI), or full methylation (both alleles methylated). The method may further comprise determining the size of the amplification products of the enriched methylated nucleic acid or unmethylated nucleic acid.

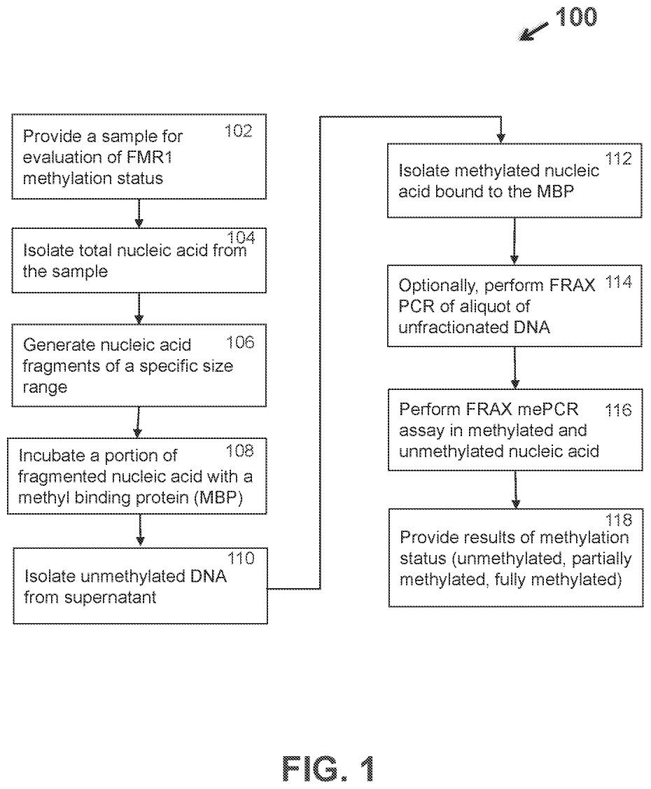

FIG. 1 shows a schematic of an embodiment of the assay method 100. Thus, the assay may comprise the step 102 of providing a sample from a subject to be screened for alleles that may present risk of fragile X (e.g., maternal and/or prenatal specimens).

The sample may be processed to isolate nucleic acid 104. In one embodiment, the nucleic acid is DNA. In an embodiment, the DNA may be cell-free DNA as for example from pre-natal testing. Samples that may be used include, but are not limited to, whole blood, plasma, serum, amniotic fluid, amniotic fluid (AF) cell cultures, chorionic villus sampling (CVS), chorionic villus sample cell cultures, saliva and buccal samples.

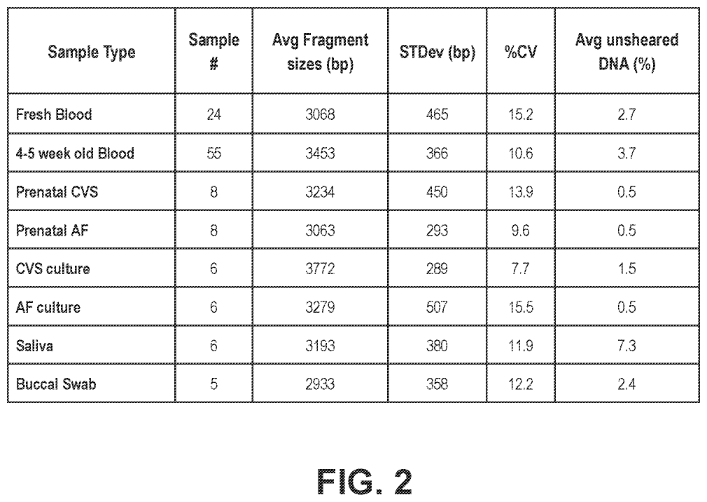

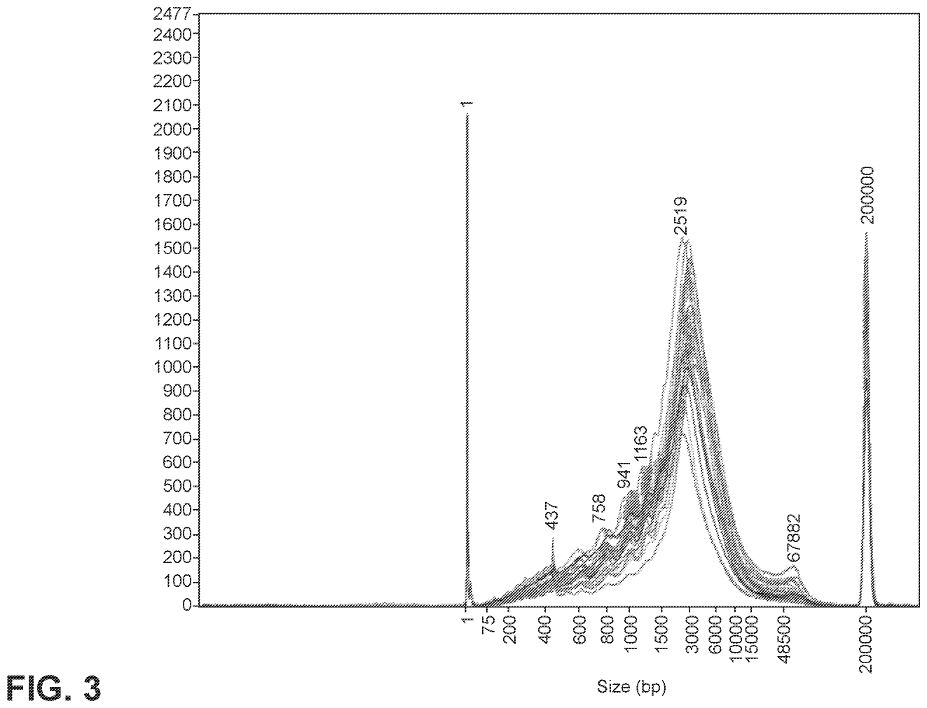

In certain embodiments, the nucleic acid is treated to generate fragments of a specific size or within a specific size range that bind efficiently to a methyl-binding protein 106. In certain embodiments, the nucleic acid is sonicated to generate fragments that are about 2.5-3 kb in size. Or, nucleic acid fragments may be generated by other methods, such as digestion with restriction enzymes. For example, conditions may be employed such that the nucleic acid is fragmented to a size ranging from 1-5 kb, or 2-4 kb, or about 2.2-3.5 kb or about 2.4-3.2, or about 2.5 or 3 kb. In an embodiment, the optimal size is about 2.5 kb. In other embodiments, the optimal size is about 3.0 kb. Thus, the optimal fragment size may vary based on the size of the PCR product that expected based on the largest FMRI allele found for the sample. Thus, in certain embodiments, selecting fragments of a specific size can increase the specificity of the assay. For example, FIGS. 2 and 3 show examples of nucleic acid (DNA) generated using a COVARIS® 3 kb multiwell sonication plate on a COVARIS® R230 sonicater. Thus, as shown in FIG. 2 DNA from fresh blood, 4-5 week old blood, prenatal CVS, prenatal AF, CVS cultured cells, AF cultured cells, saliva and buccal swabs can be reproducibly sonicated to fragments having an average size of about 3 kb. In an embodiment, shearing is highly efficient, with >90% of the DNA sheared for all samples. FIG. 3 presents an analysis of the typical sheared DNA size distribution analyzed by Agilent fragment analyzer using a High sensitivity Large Fragment Analysis Kit DNF-464. The electropherogram shows results of 24 fresh blood DNA samples.

Referring back to FIG. 1, after preparing nucleic acid fragments of the appropriate size, the nucleic acid may be incubated with a methyl-binding protein to separate nucleic acid that is methylated from nucleic acid that is not methylated 108. In certain embodiments, the methyl binding protein is crosslinked to a solid support. For example, the methyl binding protein may be cross-linked to a hydrophilic magnetic bead. In certain embodiments, the methyl binding protein is provided as the EpiMark® Methylated DNA Enrichment Kit, from New England Biolabs (Ipswich, MA) and used according to the manufacturer's instructions. Or, other methods of enriching methylated and unmethylated nucleic acids may be used.

Next, in certain embodiments, the supernatant enriched for unmethylated nucleic acid may be collected 110 prior to washing the complexed methylated nucleic acid: methyl binding protein. After washing the MBP:methlyated nucleic acid complexes, the nucleic acid fraction enriched for methylated nucleic acid is eluted from the MBP 112. In certain embodiments, the methylated DNA is eluted in 30 μL of 10 mM Tris-HCL, pH 8.0 and can be stored at −20° C. for about up to a week.

In certain embodiments, PCR amplification is used to determine the extent of CGG expansion using a second aliquot of the total unsheared DNA 114. This can be done to verify results from the initial screening of the sample. Or in an embodiment, an aliquot of the total sheared DNA (not fractionated for either methylated or unmethylated) may be used. This may be done in parallel with meFRAX PCR 116. Thus, as discussed in more detail herein, amplification of an unmethylated FMRI locus may use primers FRAX-F1 and FRAX-R-6FAM (i.e., SEQ ID NO: 1 and SEQ ID NO: 2, respectively). Amplification of a methylated FMRI locus may use primers FRAX-F1 and FRAX-R-6HEX (i.e., SEQ ID NO: 1 and SEQ ID NO: 3, respectively). Amplification of total (unfractionated DNA) may use the same primers as used for unmethylated DNA, i.e., primers FRAX-Fl and FRAX-R-6FAM (i.e., SEQ ID NO: 1 and SEQ ID NO: 2, respectively).

Thus, the FRAX mePCR assay may be used then used to: (a) assess the methylation status of each FMRI allele detected in the sample, and optionally, (b) confirm the initial GS-PCR results from the FRAX PCR assay 116. The results may then be provided to the subject or the subject's health care provider 118.

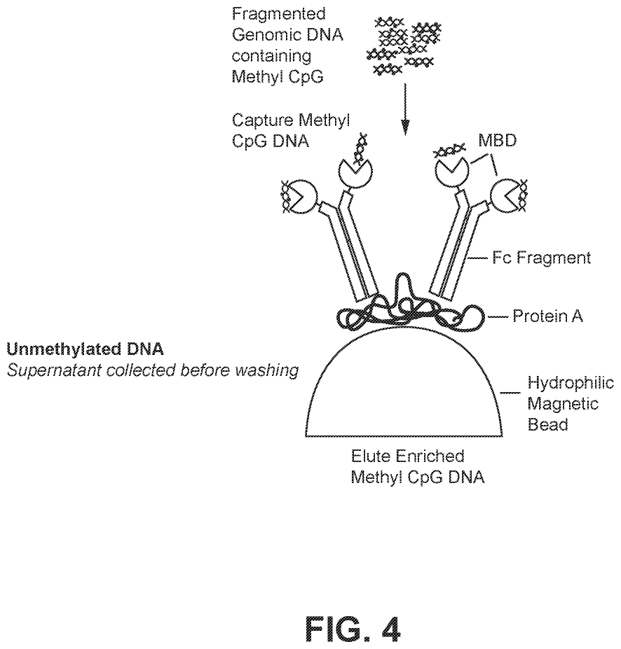

Thus, in certain embodiments, the FRAX mePCR assay starts with enrichment of unmethylated and methylated DNA from total genomic DNA that has been fragmented by sonication. As shown in FIG. 4, the enrichment process may involve specific methylated DNA binding by methyl CpG binding domain (MBD)-containing protein MBD2a (Gebhard et al., Rapid and sensitive detection of CpG-methylation using methyl-binding (MB)-PCR, Nucleic Acids Res. 2006; 34: e82; Schilling et al. 2007, Comparative analysis of tissue-specific promoter CpG methylation, Genomics 2007; 90: 314-23.). In certain embodiments, the methyl binding protein is provided as the EpiMark® Methylated DNA Enrichment Kit, from New England Biolabs (Ipswich, MA). In an embodiment, prepared MBD-bound magnetic beads can be stored at 4° C. for about 1 week.

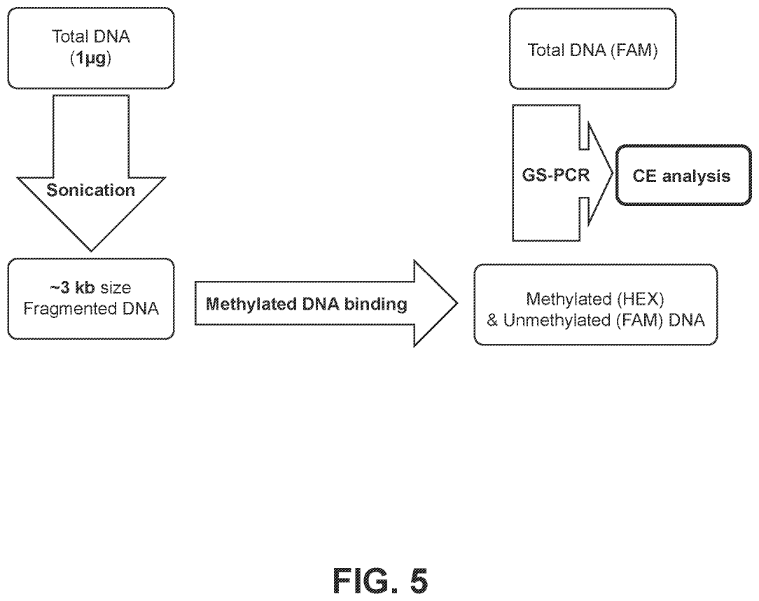

FIG. 5 shows another embodiment of a workflow for a method of the disclosure. In certain embodiments, both methylated and unmethylated DNA fractions from the same sample are analyzed by gene-specific PCR (GS-PCR) and capillary electrophoresis (CE). To ensure that the two fractions are correctly differentiated and visualized during analysis, methylated and unmethylated PCR products are end-labeled with different fluorophores; i.e., HEX and FAM, respectively. The capillary electrophoresis (CE) conditions used for the FRAX mePCR assay may be the same as in the FRAX PCR assay for both the short (GS-S) and long (GS-L) injections to resolve CGG repeat sizes ranging from normal lengths to full mutations (>200 CGGs). In certain embodiments, the peak calling tool (i.e., software, such as but not limited to GeneMapper software) used to detect peaks from CE for GS-PCR in the FRAX PCR assay is also used in the FRAX mePCR assay to detect all FMRI allele sizes found in the unfractionated total DNA sample. Methylation status of each FMRI allele is then assessed based on whether the alleles detected with total DNA are also identified in the methylated and unmethylated DNA fractions.

Still referring to FIG. 5, in certain embodiments, and as noted above, a portion of the nucleic acid (e.g., 1 μg DNA) is sheared (e.g., sonicated) to generate fragments of a specific size or within a specific size range that bind efficiently to a methyl-binding protein. In certain embodiments, 75 μL of 200 μL fragmented DNA can be used and the remainder stored at −20° C. for at least a week. In certain embodiments, fragments of about 3 kb are used. Both fractions (methylated and unmethylated) are then analyzed by gene-specific PCR (GS-PCR) (also termed me-FRAX PCR). To ensure that the two fractions are correctly differentiated and visualized during analysis, methylated and unmethylated PCR products are end-labeled with different fluorophores (e.g., HEX and FAM, respectively). Thus, the GS-PCR primers for unmethylated PCR may be SEQ ID NOs: 1 and 2 and the GS-PCR primers for methylated PCR may be SEQ ID NOs: 1 and 3. As noted herein, a second aliquot of the total DNA (i.e., not sheared and not fractionated into methylated or unmethylated) may be amplified with primers of SEQ ID NOs: 1 and 2 to determine the size of the repeat regions (i.e., FRAX PCR). In this way, results of the “total” GS-PCR should be concordant with the initial screening using FRAX PCR.

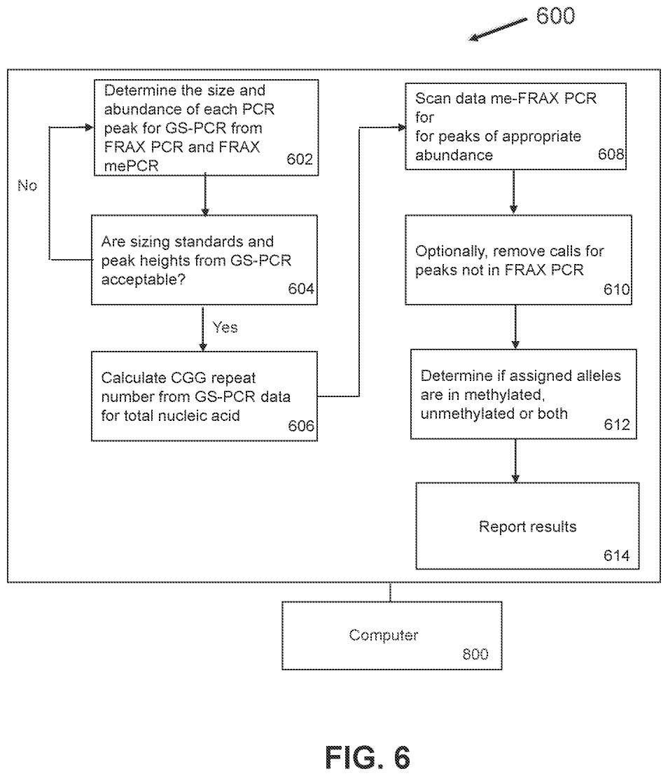

In certain embodiments, after data collection, e.g., with the 3730x1 Data Collection v.3.0 software or other analytical data processing software, the CE data for GS-PCR are analyzed using the GeneMapper v.4.0 software (ABI/Thermo Fisher) or other analytical data processing software. An embodiment of an analysis method 600 that may be used is illustrated in FIG. 6. Thus, in certain embodiments, the software outputs the analyzed size and abundance of each PCR amplicon as peak size and height, respectively, which are then processed by the FRAX mePCR methylation calling tool 602. Thus, in certain embodiments, the mePCR algorithm first checks the sizing standards and peak heights to ensure that the GS-PCR CE data quality for FRAX PCR with the total DNA as well as the FRAX mePCR are acceptable for analysis 604. Then, the method may use the sample's GS-PCR peak data derived from total DNA to calculate the CGG repeat number for each allele 606. Using these CGG repeat numbers, the algorithm may then scan the GS-PCR CE data of the methylated and unmethylated DNA fractions in the

HEX and FAM channels, respectively, to identify corresponding peaks that are above the allele-specific peak height thresholds 608. In an embodiment, besides those detected using total DNA, the methylation status calling algorithm will not identify additional peaks in the methylated or unmethylated fraction 608. In this way, the algorithm functions similarly to the process used currently with Southern Blot (SB) analysis in which the CGG alleles detected by GS-PCR in the FRAX PCR assay are confirmed and the methylation status determined. Detection of the same peak in total DNA as well as in either the methylated or unmethylated fraction, but not both, indicates fully methylated or unmethylated status, respectively, for the allele. If the same peak occurs in both methylated and unmethylated fractions as well as total DNA, the allele is called as partially methylated 612. At this point, the results may be reported 614.

As discussed herein, each of these analysis steps may be controlled by a computer or data processor 800 using a non-transitory computer readable storage medium containing instructions which, when executed on a data processor, cause the data processor to perform any one of these steps.

### Compositions and Kits

Also disclosed herein are compositions and kits for performing any of the disclosed methods or running any of the components and/or stations of the disclosed systems. In an embodiment, the composition may comprise an oligonucleotide having the sequence as set forth in SEQ ID NOs. 1 or 4. In an embodiment, the oligonucleotide having the sequence as set forth in SEQ ID NOs. 1 or 4 may be labeled with a detectable moiety. For example, in certain embodiment the composition may comprise at least one primer having the sequence of SEQ ID NOs: 1, 2, 3 or 4.

Also disclosed are kits for performing any of the preceding or subsequent method embodiments or using any of the compositions of any of the preceding or subsequent composition embodiments. In an embodiment, the kit may comprise an oligonucleotide having the sequence as set forth in SEQ ID NOs. 1 or 4. In an embodiment, the oligonucleotide having the sequence as set forth in SEQ ID NOs. 1 or 4 may be labeled with a detectable moiety. For example, in certain embodiment the kit may comprise at least one primer having the sequence of SEQ ID NOs: 1, 2, 3 or 4. The kit may further comprise nucleic acid molecules that provide positive and/or negative controls for assaying the FMRI gene and/or methylation status of the FMRI gene. Thus, the kit may comprise a nucleic acid having an unexpanded FMRI repeat region (i.e., <55 CGG repeats) as a negative control. Additionally and/or alternatively, the kit may comprise as a positive control, a nucleic acid having an expanded FMRI repeat region, e.g., a premutation (PM) of about 55-200 CGG repeats, and/or full mutation (FM) of >200 CGG repeats alleles. The negative and/or positive controls may be pre-characterized as being fully methylated, partially methylated or unmethylated. The kit may further comprise reagents for enrichment of methylated and/or unmethylated nucleic acid. For example, the kit may comprise a methyl binding protein. The methyl binding protein may comprise (i) an Fc portion of an antibody; (ii) a short flexible peptide linker; and (iii) a DNA-binding domain of an MBD2 protein. The kit may further comprise instructions for use.

### Systems for FMRI Methylation Assay

Also, disclosed are systems for detecting methylation of the fragile X FMRI gene. In certain embodiments, the system may perform any one of the steps of the disclosed methods. The system may be embodied in a variety of ways. Further, each of the stations and/or components described herein may be a separate station or component or may be combined and/or controlled by a different station or component.

For example in certain embodiments, the system may comprise a station or component for isolating total nucleic acid from the sample. Also, the system may comprise a station or component for fragmenting the nucleic acid to a specific size to optimize binding of methylated nucleic acids to a MBP. For example, conditions may be employed such that the nucleic acid is fragmented to a size ranging from 1-5 kb, or 2-4 kb, or about 2.5-3.5 kb. The system may comprise a component or station for enriching methylated and/or unmethylated nucleic acid. For example, the system may further comprise a component or station for contacting a first portion of the isolated total nucleic acid with a methyl binding protein. The system may further comprise a component(s) or a station(s) to collect fractions of the nucleic acid that are enriched for methylated nucleic acid and/or unmethylated nucleic acid. The system may further comprise a station or component to perform PCR amplification of the FMRI gene. In an embodiment, the amplification is FRAX mePCR to determine if FMRI gene sequences are present in a nucleic acid fraction enriched for methylated nucleic acid and/or a nucleic acid fraction enriched for unmethylated nucleic acid or both.

The system may further comprise a component or station for determining whether an expanded allele detected in total nucleic acid from the subject is methylated or unmethylated. Thus, in certain embodiments, the system comprises software (and/or a data processor) that outputs the analyzed size and abundance of each PCR amplicon from FRAX PCR and/or meFRAX PCR as peak size and height, respectively, which are then processed by a FRAX mePCR methylation calling software tool. In certain embodiments, the mePCR algorithm first checks the sizing standards and peak heights to ensure that the FRAX PCR and/or meFRAX PCR CE data quality are acceptable for analysis. Then, in certain embodiments, the methods may use the sample's GS-PCR peak data derived from total DNA to calculate the CGG repeat number for each allele. Using these CGG repeat numbers, the algorithm may then scan the GS-PCR CE data of the methylated and unmethylated DNA fractions in the HEX and FAM channels, respectively, to identify corresponding peaks that are above the allele-specific peak height thresholds. In an embodiment, besides those detected using total DNA, the methylation status software will not identify additional peaks in the methylated or unmethylated fraction. In this way, the CGG alleles detected by GS-PCR in the FRAX PCR assay from total nucleic acid are confirmed and the methylation status determined. Detection of the same peak in total DNA as well as in either the methylated or unmethylated fraction, but not both, indicates fully methylated or unmethylated status, respectively, for the allele. If the same peak occurs in both methylated and unmethylated fractions as well as total DNA, the allele is called as partially methylated. The system may further comprise a device for reporting the results to the subject and/or his or her healthcare provider.

Also included as part of the disclosed systems are reagents used for performing any of the steps of the method. For example, in certain embodiments, the reagents comprise at least one primer having the sequences of SEQ ID NO: 1-3 disclosed herein.

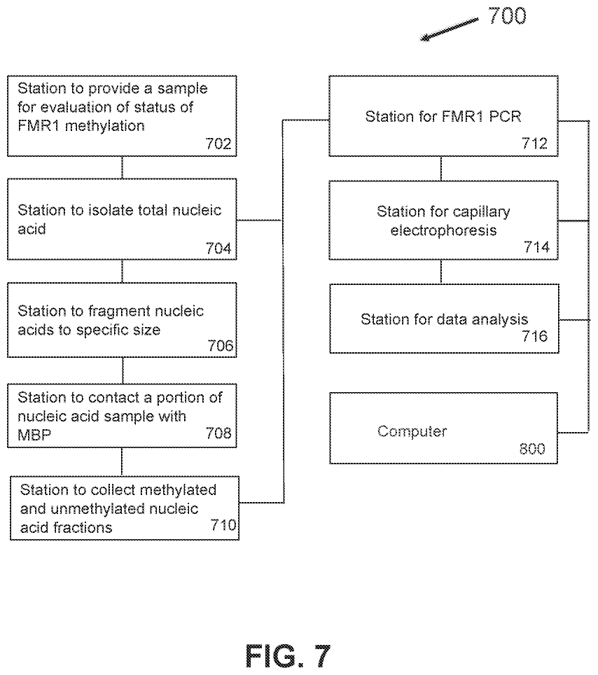

For example, as shown in FIG. 7, the system 700 may comprise a station or a component for obtaining or processing a sample for evaluation of FMRI expansion and/or methylation in a sample from a subject 702.

The system may further comprise a station or component for isolating total nucleic acid from the sample 704. Also, the system may comprise a station or component for fragmenting the nucleic acid to a specific size. In certain embodiments, the nucleic acid may be fragmented to a size to optimize binding of methylated nucleic acids to a MBP 706. In certain embodiments, the nucleic acid is sonicated to generate fragments that are about 3 kb in size. For example, conditions may be employed such that the nucleic acid is fragmented to a size ranging from 1-5 kb, or 2-4 kb, or about 2.2-3.5 kb or about 2.4-3.2, or about kb or about 3 kb.

The system may further comprise a component(s) or a station(s) to enrich the nucleic acid for methylated and/or unmethylated nucleic acid and to collect fractions of the nucleic acid that are enriched for methylated nucleic acid and/or unmethylated nucleic acid. For example, after preparing nucleic acid fragments of the appropriate size, the nucleic acid may be incubated with a methyl-binding protein to separate nucleic acid that is methylated from nucleic acid that is not methylated 708. In certain embodiments, the methyl binding protein is crosslinked to a solid support. For example, the methyl binding protein may be cross-linked to a hydrophilic magnetic bead. Next, in certain embodiments, the supernatant containing mostly unmethylated nucleic acid may be collected prior to washing the complexed methylated nucleic acid: methyl binding protein and isolating the methylated nucleic acid as disclosed herein. Thus, the system may comprise a station or component for collecting fractions enriched for methylated and/or unmethylated nucleic acid 710.

The system may further comprise a station or component to perform PCR amplification of the FMRI gene 712. In an embodiment, the amplification is FRAX mePCR to determine if FMRI gene sequences are present in a nucleic acid fraction enriched for methylated nucleic acid and/or a nucleic acid fraction enriched for unmethylated nucleic acid or both. Additionally, the station or component may comprise FRAX PCR to determine the size of an FMRI gene alleles in a subject, where the assay optionally includes a determination of the number of expanded CGG repeats and a determination of the sex of the subject from whom the sample was obtained.

In certain embodiments, the PCR amplification products from FRAX mePCR and/or FRAX PCR are analyzed by capillary electrophoresis (CE) or other size separation step. Thus, the system may comprise a component or a station for CE 714.

Analysis of the data may be performed using automated analysis systems such as those disclosed herein. Thus, the system may comprise a station or a component for data analysis 716.

As illustrated in FIG. 7, any of the stations and/or components of the system may be automated, robotically controlled, and/or controlled at least in part by a computer (e.g., data processor) 800 and/or programmable software. For example, the station(s) and/or components(s) for FRAX mePCR or FRAX PCR, or CE, or data analysis may be controlled by a computer. Thus, the system may comprise a computer-program product tangibly embodied in a non-transitory machine-readable storage medium, including instructions configured to run the system or any part (e.g., station or component) of the system and/or perform a step or steps of the methods of any of the disclosed embodiments. In some embodiments, a system is provided that includes one or more data processors and a non-transitory computer readable storage medium containing instructions which, when executed on the one or more data processors, cause the one or more data processors to perform part or all of one or more methods or processes disclosed herein and/or run any of the parts of the systems disclosed herein.

For example, disclosed is a system comprising one or more data processors, and a non-transitory computer readable storage medium containing instructions which, when executed on the one or more data processors, cause the one or more data processors to perform actions to direct at least one of the steps of determining methylation of an FMRI gene in a sample from a subject comprising the steps of: isolating total nucleic acid from the sample, the total nucleic acid comprising both methylated nucleic acid and unmethylated nucleic acid; contacting a first portion of the isolated total nucleic acid with a methyl binding protein; isolating a portion of the unbound nucleic acid, thereby generating a nucleic acid fraction enriched for unmethylated nucleic acid; isolating the bound nucleic acid, thereby generating a nucleic acid fraction enriched for methylated nucleic acid; conducting PCR amplification of the FMRI gene using an aliquot of nucleic acid from the fraction enriched for methylated nucleic acid; conducting PCR amplification of the FMRI gene using an aliquot of nucleic acid from the fraction enriched for unmethylated nucleic acid; and determining the presence or absence of an FMRI amplification product in both the fraction enriched for methylated nucleic acid and the fraction enriched for unmethylated nucleic acid.

The data processor and non-transitory computer readable storage medium may further comprise instructions for analyzing the data from FRAX me-PCR. Thus the data processor may comprise a non-transitory computer readable storage medium containing instructions to perform the steps of providing the size and abundance of each PCR amplicon from FRAX and/or meFRAX PCR as peak size and height. In certain embodiments, the analysis checks the sizing standards and peak heights to ensure that the GS-PCR CE data quality are acceptable for analysis. The method may then use the sample's GS-PCR peak data derived from FRAX and/or meFRAX PCR to calculate the CGG repeat number for each allele. Using these CGG repeat numbers, the method may then scan the GS-PCR CE data of the methylated and unmethylated DNA fractions in the HEX and FAM channels, respectively, to identify corresponding peaks that are above the allele-specific peak height thresholds. In an embodiment, besides those detected using total DNA, the methylation status calling algorithm will not identify additional peaks in the methylated or unmethylated fraction. In this way, CGG alleles detected by GS-PCR in the FRAX PCR assay are confirmed and the methylation status determined. Detection of the same peak in total DNA as well as in either the methylated or unmethylated fraction, but not both, indicates fully methylated or unmethylated status, respectively, for the allele. If the same peak occurs in both methylated and unmethylated fractions as well as total DNA, the allele is called as partially methylated. The system may further include instructions to report the results.

Also disclosed is a computer-program product tangibly embodied in a non-transitory machine-readable storage medium, including instructions configured to run the systems and/or perform a step or steps of the methods of any of the disclosed embodiments. For example, in certain embodiments, the computer-program product tangibly embodied in a non-transitory machine-readable storage medium includes instructions configured to cause one or more data processors to perform actions to direct at least one of the steps of determining methylation of an FMRI gene in a sample from a subject comprising the steps of: isolating total nucleic acid from the sample, the total nucleic acid comprising both methylated nucleic acid and unmethylated nucleic acid; contacting a first portion of the isolated total nucleic acid with a methyl binding protein; isolating a portion of the unbound nucleic acid, thereby generating a nucleic acid fraction enriched for unmethylated nucleic acid; isolating the bound nucleic acid, thereby generating a nucleic acid fraction enriched for methylated nucleic acid; conducting PCR amplification of the FMRI gene using an aliquot of nucleic acid from the fraction enriched for methylated nucleic acid; conducting PCR amplification of the FMRI gene using an aliquot of nucleic acid from the fraction enriched for unmethylated nucleic acid; and determining the presence or absence of an FMRI amplification product in both the fraction enriched for methylated nucleic acid and the fraction enriched for unmethylated nucleic acid. The computer-program product may further comprise instructions for analyzing the data from FRAX Me-PCR as disclosed in detail herein (see e.g., FIG. 6).

Thus, the computer program product may comprise instructions to perform the steps of providing the size and abundance of each PCR amplicon from FRAX and/or meFRAX PCR as peak size and height. In certain embodiments, the analysis checks the sizing standards and peak heights to ensure that the GS-PCR CE data quality are acceptable for analysis. The method may then use the sample's GS-PCR peak data derived from FRAX and/or meFRAX PCR to calculate the CGG repeat number for each allele. Using these CGG repeat numbers, the method may then scan the GS-PCR CE data of the methylated and unmethylated DNA fractions in the HEX and FAM channels, respectively, to identify corresponding peaks that are above the allele-specific peak height thresholds. In an embodiment, besides those detected using total DNA, the methylation status calling algorithm will not identify additional peaks in the methylated or unmethylated fraction. In this way, CGG alleles detected by GS-PCR in the FRAX PCR assay are confirmed and the methylation status determined. Detection of the same peak in total DNA as well as in either the methylated or unmethylated fraction, but not both, indicates fully methylated or unmethylated status, respectively, for the allele. If the same peak occurs in both methylated and unmethylated fractions as well as total DNA, the allele is called as partially methylated. The system may further include instructions to report the results.

Thus, the systems and computer products may perform any of the methods or steps of the methods disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

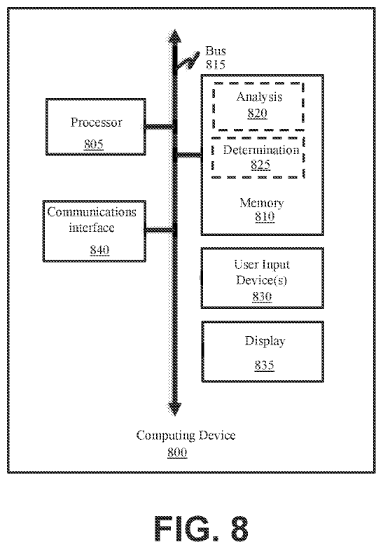

FIG. 8 shows a block diagram of an analysis system 800 used for detection and/or quantification of a progesterone metabolite. As illustrated in FIG. 8, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 8, modules, engines, or components are shown for implementing the methods or running any of the systems of the disclosure.

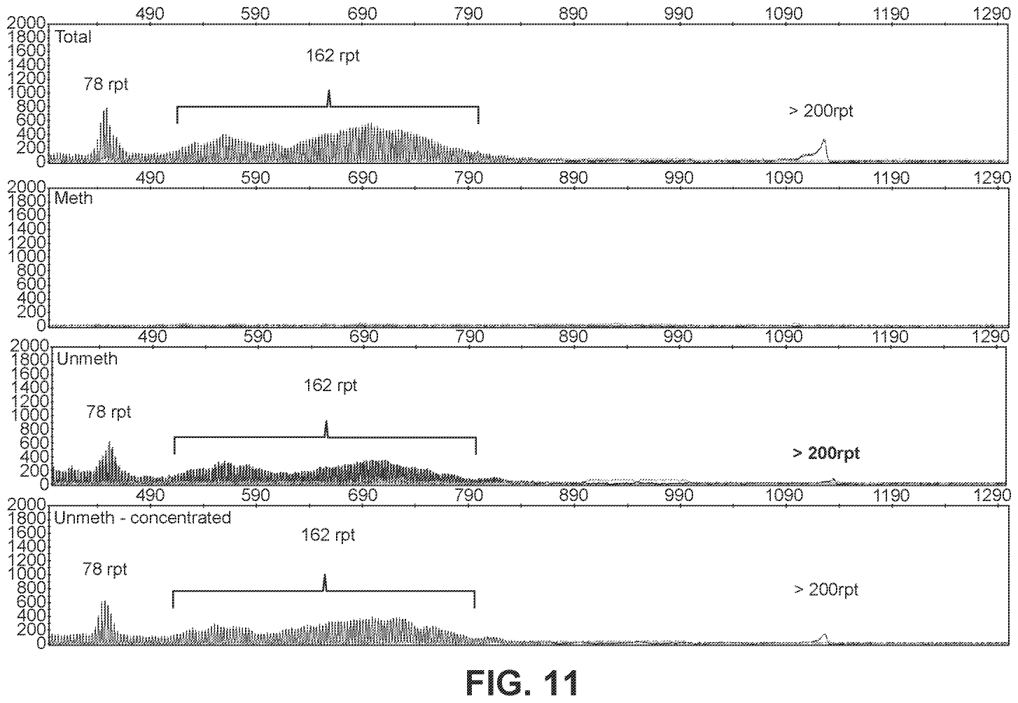

Thus, FIG. 8 illustrates an example computing device 800 suitable for use with systems and the methods according to this disclosure. The example computing device 800 includes a processor 805 which is in communication with the memory 810 and other components of the computing device 800 using one or more communications buses 815. The processor 805 is configured to execute processor-executable instructions stored in the memory 810 to perform one or more methods or operate one or more stations for detecting methylation status of FMRI alleles according to different examples, such as those in FIG. 1-7 or 9-11 or disclosed elsewhere herein. In this example, the memory 810 may store processor-executable instructions 825 that can analyze 820 results for sample as discussed herein.

The computing device 800 in this example may also include one or more user input devices 830, such as a keyboard, mouse, touchscreen, microphone, etc., to accept user input. The computing device 800 may also include a display 835 to provide visual output to a user such as a user interface. The computing device 800 may also include a communications interface 840. In some examples, the communications interface 840 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

Embodiments of the disclosed methods, compositions, kits and systems provide a high-throughput assay for characterization of the methylation status of FMRI alleles in subjects in need thereof. Thus, in certain embodiments, analytical sensitivity and specificity were >95%, or >98%, or 100%. Thus, in certain embodiments, all samples result in a methylation status call, and no false negative or false positive calls are made by the methylation status calling algorithm.

Additionally, intra-assay repeatability and/or inter-assay reproducibility for methylation status are >95%, or >98% or 100%. Also, in certain embodiments, the FRAX mePCR methylation calling algorithm disclosed herein is 100% concordant with calls made by manual analysis for methylation status. The disclosed methods may provide semi-quantitative results. For example, in certain embodiments, while the recommended DNA input for FRAX mePCR assay is 1 μg, higher or lower concentrations (e.g., about 0.2 μg) may be used. Additionally, assay performance remains robust for DNA extracted from blood specimens that had been stored at 4° C. for >60 days, and buccal swab specimens stored at −20° C. for >20 days.

The disclosed methods, compositions, kits and systems improve sample processing time and throughput significantly. For example, up to 95 samples can be analyzed and results provided by the assay in 2 days. In contrast, one SB gel can analyze ˜30 samples at a time (one gel), and takes about 1 week to get final results.

### EXAMPLES

The disclosure may be better understood by reference to the following non-limiting Examples.

### Example 1—Determination of CGG repeats from Total DNA by FRAX PCR

The evaluation of samples may involve separate assays.

1. Gene-specific PCR using primers (FRAX-F 1 and FRAX-R-6FAM) that are outside of the repeat region to determine the length of a repeated CGG trinucleotide sequence in the FMRI gene. This is used to detect large CGG repeats. Using an ABI 3730x1 Genetic Analyzer, products are sized according to the number of base pairs.

2. Non-anchored triplet-primed PCR to detect expanded alleles of the FMRI gene. This PCR uses a primer that binds to the CGG region and one of the gene-specific PCR primers (FRAX-R-6FAM).

3. Gender detection PCR is performed for interpretation of results by amplifying an X-Y homologous region in the amelogenin gene which yields a 212 bp X specific fragment and a 218 bp Y specific fragment.

The primers used for Gene-specific PCR are shown in the Table below.

TABLE 3
FRAX Specific primers

| SEQ ID | Primer | |
|---|---|---|
| NO. | Name | Sequence (5′-3′) |
| 1 | FRAX-F1 | 5′-GCT CAG CTC CGT TTC |
| | | GGT TTC ACT TCC GGT-3′ |
| 2 | FRAX-R- | 5′-FAM-AGC CCC GCA CTT |
| | 6FAM | CCA CCA CCA GCT CCT CCA-3′ |

Greater than 200 CGG repeats is considered a mutation. A premutation (PM) is 55-200 CGG repeats. Peaks are identified by scanning for first and second largest peaks (i.e., length of PCR product) of greater than a predetermined intensity (i.e., amount). Greater than 200 CGG repeats is considered a mutation. A premutation (PM) is 55-200 CGG repeats.

### Example 2—Analysis of Methylated vs. Unmethylated Nucleic Acid by meFRAX PCR

This assay uses the same primer sequences and assay conditions deployed for Fragile X gene-specific PCR (GS-PCR) assay (Example 1) to amplify the FMRI CGG-rich promoter region. However, the methylation PCR assay differs from the GS-PCR assay in that unmethylated DNA and methylated DNA are amplified in separate reactions such that the products are differentiated by the fluorescent dye that is used to label the reverse primer. The PCR products are then combined, and resolved by capillary electrophoresis. The presence or absence of GS-PCR products using methylated DNA and unmethylated template indicates the methylation status of the corresponding FMRI allele. See Tables 1 and 2 for primers used.

### Example 3—Methods

Whole blood, amniotic fluid, amniotic fluid cell cultures, chorionic villus sampling, chorionic villus sample cell cultures, saliva and buccals were validated as the specimen types for this assay. Each clinical test included one No Template Control (NTC) and three methylation controls (see below). To eliminate slight run-to-run variations in amplicon sizing of the GS-PCR products by CE, especially for highly repetitive GC-rich regions that can ultimately affect methylation status calling, the FRAX mePCR assay is run with total DNA and the methylated and unmethylated DNA fractions for a sample on the same plate. This configuration also facilitates data visualization and analysis.

### 1) Sample DNA Normalization

For total DNA GS-PCR, DNA samples were diluted to 10 ng/μL in 10 mM Tris-HCl, pH 8.0. For unmethylated and methylated DNA enrichment, DNA samples are normalized to 1 μg in 200 μL 10 mM Tris-HCl, pH 8.0.

### 2) Preparation of MBD-bound Protein A Magnetic Beads

MBD2a-Fc protein was mixed with Protein A magnetic beads for 15 min at room temperature on a tube rotator. The MBD2-Fc/Protein A magnetic bead mixture is stable for up to 1 week at 4° C.

### 3) Sonication

Normalized DNA samples (1 μg) were sheared into 3 kb fragments using a Covaris S2 sonicator. The fragmented DNA samples were transferred to fresh tubes or plates, and can be stored at −20° C. for up to a week.

### 4) Enrichment of Methylated/Unmethylated DNA

To each well of a 96-well plate, 20 μL 1X Bind/Wash Reaction Buffer and 4 μL MBD2-Fc/Protein A magnetic bead mixture was added followed by 76 μL of fragmented DNA sample (100 μL total). Immunoprecipitation of the methylated DNA was performed on a plate thermomixer for 20 min at room temperature. Once binding was completed, the beads were pulled down on a magnetic stand and the supernatant, which contained the unmethylated DNA fraction, was collected from each well and saved; it is stable at −20° C. for up to a week. After three 3-min washes, the methylated DNA fraction from each sample was eluted with 30 μL 10 mM Tris-HCl, pH 8.0 in a thermomixer at 65° C. for 15 min. These samples were stable at −20° C. for up to a week.

### GS-PCR Amplification

Amplification was carried out on a 96-well PCR plate that was evenly divided into three sections. Columns 1-4, 5-8, and 9-12 were assigned to the total, unmethylated and methylated DNA, respectively, such that the same set of samples were tested in parallel. Controls were loaded in the first 4 wells followed by clinical samples (run capacity=28 samples+4 controls). Four microliter DNA samples (from steps 1 & 4) were added to 26 μL PCR reaction cocktail. PCR conditions are shown in Table 4. PCR cycling time: 30 cycles, 4.5 hrs.

TABLE 4
Temperature control mode: Calculated
Lid control mode: Tracking at 10° C. above

| CYCLES | Temperature (° C.) | TIME | | FUNCTION |
|---|---|---|---|---|
| 1 | 96 | 3 | min | Denaturation of dsDNA |
| 15 | 98.5 | 5 | sec | Denaturation of dsDNA |
| | 61 | 1 | min | Annealing |
| | 69 | 6 | min | Extension |
| 15 | 98.5 + 0.1° C./cycle | 5 | sec | Denaturation of dsDNA |
| | 61 | 1 | min | Annealing |
| | 69 | 6 | min | Extension |
| 1 | 69 | 10 | min | Final extension |

| | | | |
|---|---|---|---|
| 1 | 8 | ∞ | End |

### 6) Capillary Electrophoresis

Long and short injections of the PCR products were resolved on an ABI 3730x1 fragment analyzer.

### 7) Data Analysis

The electrophoresis data were analyzed using GeneMapper v.4.0 software and a custom methylation status calling algorithm.

### 8) Assay Controls

1) No Template Control (NTC): 10 mM Tris-HCl, pH 8.0 (cocktail blank) is used in place of a DNA sample to ensure there is no reagent contamination throughout the process.

2) Unmethylated allele control (C1): GM20230 (Coriell; CDC genetic testing reference material (Amos Wilson et al. 2009, Consensus characterization of 16 FMRI reference materials: a consortium study. J Mol Diagn. 2008; 10:2-12), a male cell line DNA carrying a 53-55 CGG repeat allele. This allele is unmethylated and should be detected in both Total DNA and unmethylated DNA.

3) Fully methylated allele control (C2): GM09237 (Coriell) male cell line DNA carrying a 931-940 CGG repeat allele. The allele is detected as a >200 CGG repeat allele in both Total DNA and methylated DNA, and undetectable in unmethylated DNA fraction.

4) Partially methylated allele control (C4): A female blood DNA sample carrying normal repeat size alleles. Both alleles are detected in total, unmethylated and methylated DNA.**

The methylation status calling algorithm produces two possible results: (1) the methylation status of each allele or (2) a sample that is flagged for manual review (Table 5).

TABLE 5
Methylation status

| Allele detected | | |
|---|---|---|
| Methylated DNA | Unmethylated DNA | Methylation status |
| Yes | Yes | Partially methylated |
| Yes | No | Fully methylated |
| No | Yes | Unmethylated |
| No | No | Flag for Manual Review |

### Example 4—Validation

A total of 26 samples were included in the validation study and consisted of five different sample types: 6 blood samples that are collected in either ACD (yellow top) or EDTA (lavender top) blood tubes, 5 amniotic fluid (AF) direct or culture samples, 5 chorionic villus sampling (CVS) direct or culture samples, 5 saliva samples, and 5 buccal swab samples. All of them had Southern Blot (SB) results as confirmation for the methylation status calls. The types and number of samples used for validation are listed in Table 6 below. In Table 5, PM =premutation and FM=full mutation.

TABLE 6

| | Types and numbers of | |
|---|---|---|
| Experiment | samples | Variants |
| Inter- and intra- | 10 samples, ≥1 sample | ≥1 each: FM male, |
| assay | from each sample type | FM female, FM or PM |
| reproducibility | (Blood, Prenatal - AF and | mosaic methylation, |
| | CVS direct and cultured, | Normal female, |
| | saliva, buccal swab) | Normal male |
| Sensitivity and | 26 samples, including 6 | ≥2 each: FM male, |
| Specificity | blood samples and 5 | FM female, FM or PM |
| | samplesfor each other | mosaic methylation, |
| | sample type | Normal female, |
| | (Prenatal - AF and | Normal male |
| | CVS direct or cultured, | |
| | saliva, buccal swab) | |
| DNA Input | 3 blood and 1 AF | ≥1 each: FM male, |
| tolerance | culture samples | FM female, FM or PM |
| | | mosaic methylation |
| Specimen | 4 samples, | N/A |
| Stability | including blood and | |
| Tolerance | buccal swab sampletypes | |

Intra-assay reproducibility for methylation status calls by the FRAX mePCR calling algorithm was 100% based on 60 alleles. For inter-assay reproducibility, the methylation status calls were reproducible for 59 alleles (98.3%) with only one allele in one CVS replicate sample called differently. Minor allele sizing differences among replicates are expected due to experimental variation, especially for highly repetitive GC-rich regions resolved by CE. In addition, the resolution of CGG repeat number determined by GS-PCR was previously validated to vary by 1-4 repeats, depending on the length of the repeats. Therefore, given the inherent run-to-run variations, a difference of 1-2 repeats (i.e., 3-6 bases) among replicates is within the expected variation for a normal or expanded allele.

1) Analytical Sensitivity and Specificity

Analytical sensitivity and specificity were evaluated using 26 DNA samples, including 6 blood samples that are collected in either ACD (yellow top) or EDTA (lavender top) blood tubes, amniotic fluid (AF) direct or cultured samples, 5 chorionic villus sampling (CVS) direct or cultured samples, 5 saliva samples, and 5 buccal swab samples.

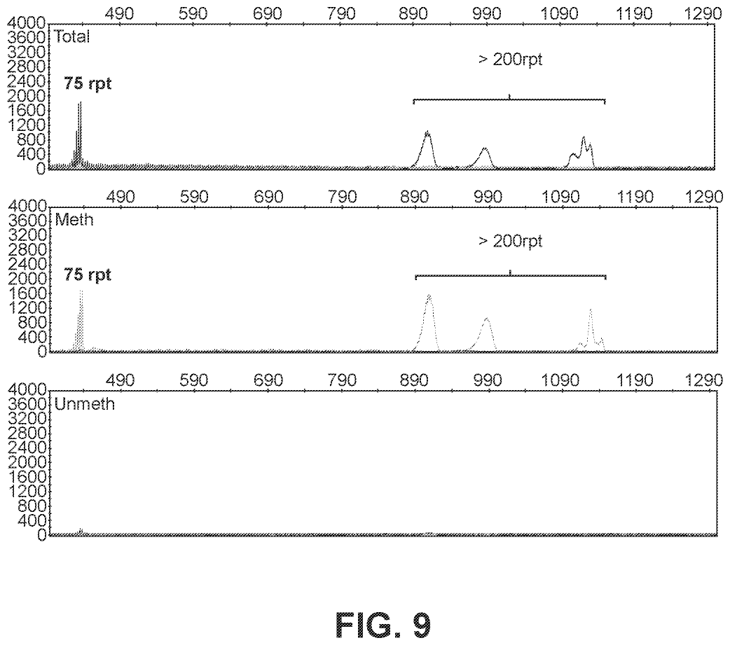

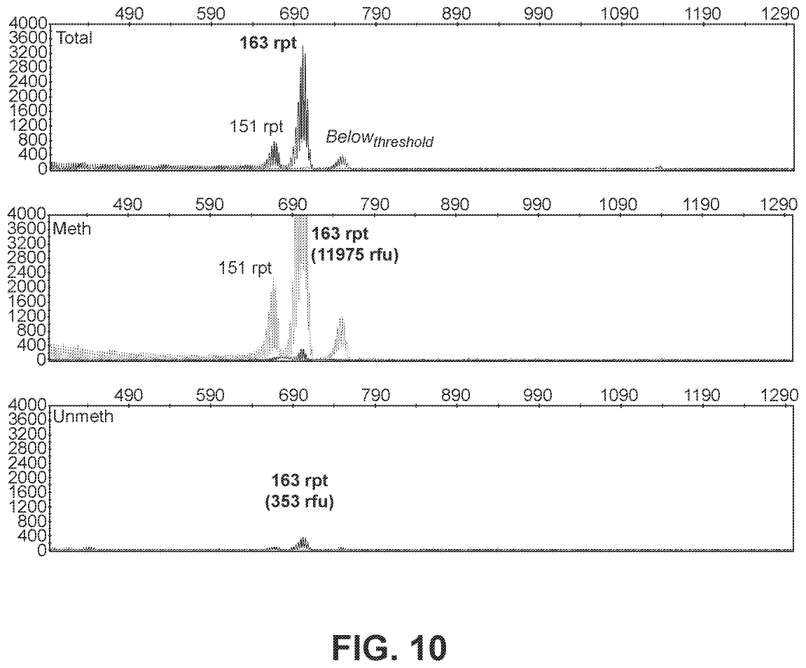

The FRAX mePCR assay determined the methylation status of all alleles for all samples. All controls passed and no false positives or false negatives were called. Overall analytical sensitivity and specificity were 100% after manual review based on 98 alleles (including mosaic samples). One additional allele in each of two mosaic samples, AFC4 (FIG. 8) and BL2 (FIG. 9), had inconclusive results by Southern analysis, and another mosaic sample, BL7 (FIG. 10), was flagged by the methylation status calling algorithm for manual review.

For AFC4, the sample was not flagged for manual review since all alleles identified with Total DNA by GS-PCR could be accounted for in the methylated and unmethylated fractions. However, when the mePCR results were compared to the SB data, we found that the 75 CGG allele, which is fully methylated based on the mePCR analysis (FIG. 8), was not detected in the Southern Blot (SB) assay as either a 3.0 kb or 5.4 kb band that corresponded to an unmethylated or methylated allele, respectively. This allele appears to be a minor one in the mosaic sample, and although it was identified by both GS-PCR and mePCR, it is likely that itsabundance in the sample is below the detection limit for autoradiography after overnight exposure. Importantly, the clinically significant methylation status of the full mutation allele at >200 CGG of AFC4 was clearly visible by both mePCR and SB analysis and concordant between the two methods.

For BL2, a 163 CGG allele peak was detected in both the methylated and unmethylated DNA fraction (FIG. 9). The SB assay also detected the methylated 163 CGG allele as a 5.7 kb band, but not the 3.3 kb band corresponding to the unmethylated allele. This is consistent with the much higher peak detected in the methylated DNA fraction compared to unmethylated DNA by mePCR (11,975 vs. 353 rfu), and indicates that the signal for the unmethylated 163 CGG allele is likely below the detection limit for the SB assay. For either AFC4 or BL2, longer exposures were not available to determine conclusively the methylationstatus for either of these minor alleles.

The mosaic sample, BL7, which was flagged for manual review, has an extremely unstable allele with a wide size range corresponding to ˜110-175 CGG repeats. The highest peak among the stutter peaks was sized at 162 CGG repeats by GS-PCR and detected only in the unmethylated fraction along with a 78 CGG allele (FIG. 10). The SB results showed a smear between 3.0 and 3.4 kb, which was confirmed after a 3-day autoradiographic exposure, and corresponded to unmethylated alleles with CGG repeats between 70 and 200. The methylation status calling algorithm could not detect a peak above the 100 rfu threshold for an allele at >200 CGG repeats and correctly flagged the sample for manual review. Side by side visualization of the electropherograms for Total, methylated and unmethylated PCR results found that the >200 CGG allele was only observed in total DNA and the unmethylated DNA fraction and was concordant with the Southern results. Since the unstable 162 CGG allele produced an unusually large number of stutter peaks during PCR amplification, it may have affected amplification efficiency of the large but less abundant FM allele in the unmethylated DNA fraction. A post-enrichment DNA cleanup and concentration step of the unmethylated fraction helped increase the FM allele signal to >100 rfu (FIG. 10, Unmeth—concentrated panel).

2) DNA Input Tolerance

Three blood and one prenatal AFC DNA samples were run in the study, and their concentrations measured using a NanoDrop spectrophotometer. Three decreasing input amounts from 1, 0.5, to 0.2 μg were tested in the assay and analyzed by the methylation statuscalling algorithm. Although input down to 0.2 μg still resulted in concordant calls for alleles that are either unmethylated or fully methylated, some partially methylated alleles were less tolerantat the lower input amounts, especially at 0.2 μg, resulting in discordant calls.

It was found that analysis of the mePCR CE peak height data for the two alleles affected by lowering the input revealed that the peak heights in both DNA fractions decreased proportionally with input amount, indicating the assay's semi-quantitative capability. Notably, these alleles shared a similar feature; i.e., the peak height detected in either methylated (BL3-30 rpt) or unmethylated (AFC2 FM allele) DNA was much lower than that in the other fraction. The signal differences between the two fractions could be quite substantial, e.g. at least a 10-fold difference with 1 μg input. Lowering DNA input could easily cause the peak height in the less abundant fraction to fall below the calling threshold and result in an incorrect call. Since the “Partially Methylated” call is most affected regardless of the repeat size, 1 μg input starting DNA at the sonication step may be recommended for the FRAX mePCR assay.

3) Specimen Stability Tolerance

Two whole blood specimens were stored at 4° C. for at least 27 days and extracted at two time points, one within 7 days and the other at least 27 days post-accessioning. Also included were two buccal swab specimens stored at −20° C. and extracted at two time points, one within 7 days and the other after 20 days. It was found that DNA extracted from blood samples stored up to 63 days did not adversely affect performance, and resulted in the same allele sizing and methylation status calls as blood extracted within 7 days. Similarly, DNA extracted from Ultraflock (SWU19) or cotton swab (SWC17) stored for 21 days at −20° C. produced identical results with those extracted immediately after collection. Thus, both sample types stored at conditions described above do not adversely affect assay performance.

4) Maternal Cell Contamination (MCC)

In this study, the methylation algorithm used the allele sizes determined by GS-PCR with total DNA to identify corresponding peaks in the methylated and unmethylated CE data and make the methylation status calls.

FRAX Methylation Calling Algorithm Testing

Methylation status analysis of FMRI alleles in each sample was performed by determining the CGG repeat sizes in total DNA by GS-PCR. Once the allele repeat sizes were determined, the algorithm performs a basic search and match task using the CE peak data generated with the methylated and unmethylated DNA fractions from the same sample and the alleles identified by GS-PCR with total DNA to determine the methylation status of each allele. Finally, methylation status of each allele is called based on the criteria listed above (Table 5-Methylation Status).

Based on the 26 samples run in this study, the results from the FRAX mePCR methylation calling algorithm were 100% concordant with that from manual analysis. To further the analysis of test concordance, 175 sample runs (including the same samples tested in separate runs) during assay development were also analyzed manually and by the algorithm, and the results were 100% in agreement between the two methods. Not only did the algorithm perform comparable to manual analysis, it also performed as expected in identifying any samples in which the input CE data quality fluctuated or the genotype output was out of normal range, flagging them for “Manual Review”.

6) Reporting

Typical FRAX mePCR results and interpretation are listed in Tables 7 and 8 below. Final reporting depends on gender and the number of CGG repeats per allele as determined in the FRAX PCR assay. Of note, for CVS, full mutations may not be methylated because methylation may not be fully established at the gestational period when CVS is typically performed.

TABLE 7
FRAX mePCR Results and Interpretation of Methylation Pattern

| | | Interpretation of |
|---|---|---|
| Gender | FraX mePCR Results | Methylation Pattern |
| Female | Allele completely methylated | Abnormal |
| Female | Allele partially methylated | Normal |
| Female | Allele unmethylated | Abnormal |
| Male | Allele completely methylated | Abnormal |
| Male | Allele partially methylated | Abnormal |
| Male | Allele unmethylated | Normal |

TABLE 8
Typical Results and Interpretation for
CGG Repeat Size and Methylation Pattern

| CGG Repeat | Methylation | Clinical |
|---|---|---|
| Size | Pattern | Interpretation |
| Normal | Normal | Negative |
| Range | Methylation | |
| (<55 CGG) | Pattern | |
| Premutation | Normal | Premutation carrier of fragile X syndrome |
| Range | Methylation | Females: At risk for primary ovarian |
| (55-200 | Pattern | insufficiency and late-onset fragile |
| CGG) | | X-associated tremor/ataxia syndrome |
| | | (FXTAS), and for having children |
| | | with fragile X syndrome. |
| | | Males: At risk for developing FXTAS. |
| | | Daughters of men with premutations inherit |
| | | the premutation and are at risk for FXTAS, |
| | | primary ovarian insufficiency, and for having |
| | | children with fragile X syndrome. |
| Full | Abnormal | Full mutation carrier of fragile X syndrome |
| Mutation | Methylation | Females: Approximately 50% of females |
| Range (>200 | Pattern | carrying a full mutation have fragile |
| CGG) | | X syndrome-associated symptoms. |
| | | Males: Predicted to be affected with |
| | | fragile X syndrome. |

### Conclusion

Technical performance of the FRAX Methylation PCR Assay was validated to be reproducible and robust for blood, prenatal (amniotic fluid and chorionic villus sampling and cultures thereof), saliva and buccal DNA specimens.

1) Analytical sensitivity and specificity were 100% based on 98 alleles in which all were concordant with Southern blot results after manual review. All samples resulted in a methylation status call, and no false negative or false positive calls were made by the methylation status calling algorithm. A minor allele in two mosaic samples could not be definitively resolved by Southern blot analysis although they were clearly detectable in the total DNA and either the methylated (AFC4) or unmethylated (BL2) DNA fraction by the FRAX mePCR assay.

2) Based on 10 specimens consisting of at least one blood, prenatal (cultured cells and direct), buccal and saliva specimen type, intra- assay repeatability and inter-assay reproducibility for methylation status were 100% (60/60 alleles) and 98.3% (59/60 alleles), respectively. One normal allele of a CVS replicate in an inter-assay run was called as partially methylated instead of unmethylated likely due to sampling and immature methylation of this tissue type, resulting in slight fluctuations in run-to-run peak heights when they hovered around the peak calling threshold.

3) The recommended DNA input for FRAX mePCR assay is 1 μg. Assay performance remained robust for DNA extracted from blood specimens that had been stored at 4° C. for up to 63 days, and buccal swab specimens stored at −20° C. for 21 days.

4) The FRAX mePCR methylation calling algorithm was 100% concordant with calls made by manual analysis for methylation status.

5) Not counting no-template control, up to 95 samples can be analyzed and results provided in 2 days. One SB gel can analyze ˜30 samples at a time (one gel), and can takes a week (5-6 days at least) to get final results if long film exposure is needed.

### Example 5—Illustrative Embodiments

As used below, any reference to methods or systems is understood as a reference to each of those methods or systems disjunctively (e.g., “Illustrative embodiment 1-4 is understood as illustrative embodiment 1, 2, 3, or 4.”).

Illustrative embodiment 1 is a method for determining methylation of an FMRI gene in a sample from a subject comprising the steps of:

isolating total nucleic acid from the sample, the total nucleic acid comprising both methylated nucleic acid and unmethylated nucleic acid;

contacting a first aliquot of the isolated total nucleic acid with a methyl binding protein;

isolating a portion of the unbound nucleic acid, thereby generating a nucleic acid fraction enriched for unmethylated nucleic acid;

isolating the bound nucleic acid, thereby generating a nucleic acid fraction enriched for methylated nucleic acid;

conducting PCR amplification of the FMRI gene using an aliquot of nucleic acid from the fraction enriched for methylated nucleic acid;

conducting PCR amplification of the FMRI gene using an aliquot of nucleic acid from the fraction enriched for unmethylated nucleic acid; and

determining the presence or absence of an FMRI amplification product in both the fraction enriched for methylated nucleic acid and the fraction enriched for unmethylated nucleic acid.

Illustrative embodiment 2 is the method of any preceding or subsequent illustrative method embodiment, further comprising determining the size of the FMRI amplification product in at least one of the fraction enriched for methylated nucleic acid and the fraction enriched for unmethylated nucleic acid.

Illustrative embodiment 3 is the method of any preceding or subsequent illustrative method embodiment, further comprising determining the number of CGG repeats in the FMRI gene for the amplification product in at least one of the fraction enriched for methylated nucleic acid and/or the fraction enriched for unmethylated nucleic acid.

Illustrative embodiment 4 is the method of any preceding or subsequent illustrative method embodiment, further comprising performing PCR amplification of the FMRI gene using a second aliquot of nucleic acid from the total nucleic acid and determining the size of at least one FMRI amplification product.

Illustrative embodiment 5 is the method of any preceding or subsequent illustrative method embodiment, further comprising determining the number of CGG repeats at the FMRI gene for the at least one amplification product obtained from the total nucleic acid fraction.

Illustrative embodiment 6 is the method of any preceding or subsequent illustrative method embodiment, wherein determining the size of the FMRI amplification product in at least one of the fraction enriched for methylated nucleic acid and/or the fraction enriched for unmethylated nucleic acid comprises comparing the size of the amplification products for the fraction enriched for methylated nucleic acid and the fraction enriched for unmethylated nucleic acid to the size of the at least one FMRI amplification product for total nucleic acid.

Illustrative embodiment 7 is the method of any preceding or subsequent illustrative method embodiment, wherein a size of the FMRI amplification product in at least one of the fraction enriched for methylated nucleic acid and the fraction enriched for unmethylated nucleic acid is determined to be the size of the at least one FMRI amplification product for total nucleic acid.

Illustrative embodiment 8 is the method of any preceding or subsequent illustrative method embodiment, wherein at least one primer used to generate an FMRI amplification product is labeled with a detectable moiety.

Illustrative embodiment 9 is the method of any preceding or subsequent illustrative method embodiment, wherein the at least one primer used to generate an amplification product from the FMRI gene from the fraction enriched for methylated nucleic acid and/or the fraction enriched for unmethylated nucleic acid and/or the total nucleic acid have the same sequence but are labeled with a different detectable moiety.

Illustrative embodiment 10 is the method of any preceding or subsequent illustrative method embodiment, wherein the detectable moiety is a fluorescent dye.

Illustrative embodiment 11 is the method of any preceding or subsequent illustrative method embodiment, wherein the fluorescent dye used for detection of the amplification products from the fraction enriched for unmethylated nucleic acid and/or the total nucleic acid is 6-carboxyfluorescein (FAM) and the dye used for detection of the amplification product from the fraction enriched for methylated nucleic acid is hexachlorofluorescein (HEX).

Illustrative embodiment 12 is the method of any preceding or subsequent illustrative method embodiment, wherein the isolated total nucleic acid is fragmented to fragments having a size ranging from 2.4-3.2 kilobase pairs (kb) prior to contacting the isolated total nucleic acid with the methyl binding protein.

Illustrative embodiment 13 is the method of any preceding or subsequent illustrative method embodiment, wherein the methyl binding protein is a bifunctional polypeptide comprising: (i) an Fc portion of an antibody; (ii) a short flexible peptide linker; and (iii) a DNA-binding domain of an MBD2 protein.

Illustrative embodiment 14 is the method of any preceding or subsequent illustrative method embodiment, wherein the size of the FMRI amplification product in the fraction enriched for methylated nucleic acid and/or the fraction enriched for unmethylated nucleic acid and/or the total nucleic acid is measured by capillary electrophoresis.

Illustrative embodiment 15 is the method of any preceding or subsequent illustrative method embodiment, further comprising determining whether the subject has either: (i) partial methylation of FMRI; (ii) both a methylated and unmethylated copy of FMRI, or (iii) full methylation of FMRI.

Illustrative embodiment 16 is the method of any preceding or subsequent illustrative method embodiment, further comprising determining whether the subject has either: (i) partial methylation of an expanded FMRI allele; (ii) both a methylated and unmethylated expanded FMRI allele; or (iii) full methylation of an expanded FMRI allele.

Illustrative embodiment 17 is a composition for performing any of the preceding or subsequent method embodiments.

Illustrative embodiment 18 is the composition of any preceding or subsequent illustrative composition embodiment, comprising at least one primer having the sequence of SEQ ID NOs: 1, 2, 3 or 4.

Illustrative embodiment 19 is a kit for performing any of the preceding or subsequent method embodiments or using any of the compositions of any of the preceding or subsequent composition embodiments.

Illustrative embodiment 20 is the kit of any preceding or subsequent illustrative kit embodiment, comprising at least one primer having the sequence of SEQ ID NOs: 1, 2, 3 or 4.

Illustrative embodiment 21 is the kit of any preceding or subsequent illustrative kit embodiment, comprising instructions for use.

Illustrative embodiment 22 is a system for performing any of the preceding or subsequent method embodiments or using any of the compositions of any of the preceding or subsequent composition embodiments or using any of the kits of any of the preceding or subsequent kit embodiments.

Illustrative embodiment 23 is the system of any preceding or subsequent illustrative system embodiment, further comprising:

a component and/or station for isolating total nucleic acid from the sample;

a component and/or station for fragmenting the isolated total nucleic acid to a specific size range;

a component and/or station for contacting a first portion of the fragmented nucleic acid with a methyl binding protein;

a component and/or station for collecting a fraction of the nucleic acid that is enriched for methylated nucleic acid and a fraction of the nucleic acid that is enriched for unmethylated nucleic acid;

a component and/or station to perform PCR amplification of the FMRI gene separately for the nucleic acid that is enriched for methylated nucleic acid and the fraction of the nucleic acid that is enriched for unmethylated nucleic acid; and

a component and/or station to determine if FMRI gene sequences are present in the nucleic acid fraction enriched for methylated nucleic acid and/or the nucleic acid fraction enriched for unmethylated nucleic acid or both.

Illustrative embodiment 24 is the system of any preceding or subsequent illustrative system embodiment, further comprising a component and/or station for determining the size of the FMRI amplification product in at least one of the fraction enriched for methylated nucleic acid and/or the fraction enriched for unmethylated nucleic acid and/or further comprising a component and/or station for determining the number of CGG repeats at the FMRI gene for the amplification product in at least one of the fraction enriched for methylated nucleic acid and/or the fraction enriched for unmethylated nucleic acid.

Illustrative embodiment 25 is the system of any preceding or subsequent illustrative system embodiment, further comprising a component and/or station for performing PCR amplification of the FMRI gene using an aliquot of nucleic acid from the total nucleic acid and determining the size of at least one FMRI amplification product.

Illustrative embodiment 26 is the system of any preceding or subsequent illustrative system embodiment, further comprising a component and/or station determining the number of CGG repeats at the FMRI gene for the at least one amplification product obtained from the total nucleic acid fraction.

Illustrative embodiment 27 is the system of any preceding or subsequent illustrative system embodiment, wherein determining the size of the FMRI amplification product in at least one of the fraction enriched for methylated nucleic acid and the fraction enriched for unmethylated nucleic acid comprises comparing the size of the amplification products for the fraction enriched for methylated nucleic acid and/or the fraction enriched for unmethylated nucleic acid to the size of the at least one FMRI amplification product for total nucleic acid.

Illustrative embodiment 28 is the system of any preceding or subsequent illustrative system embodiment, wherein a size of the FMRI amplification product in at least one of the fraction enriched for methylated nucleic acid and the fraction enriched for unmethylated nucleic acid is determined to be the size of the size of the at least one FMRI amplification product for total nucleic acid.

Illustrative embodiment 29 is the system of any preceding or subsequent illustrative system embodiment, wherein at least one primer used to generate an FMRI amplification product is labeled with a detectable moiety.

Illustrative embodiment 30 is the system of any preceding or subsequent illustrative system embodiment, wherein the at least one primer used to generate an amplification product from the FMRI gene from the fraction enriched for methylated nucleic acid and/or the fraction enriched for unmethylated nucleic acid and/or the total nucleic acid have the same sequence but are labeled with a different detectable moiety.

Illustrative embodiment 31 is the system of any preceding or subsequent illustrative system embodiment, wherein the detectable moiety is a fluorescent dye.

Illustrative embodiment 32 is the system of any preceding or subsequent illustrative system embodiment, wherein the fluorescent dye used for detection of the amplification products from the fraction enriched for unmethylated nucleic acid and/or the total nucleic acid is 6-carboxyfluorescein (FAM) and the dye used for detection of the amplification product from the fraction enriched for methylated nucleic acid is hexachlorofluorescein (HEX).

Illustrative embodiment 33 is the system of any preceding or subsequent illustrative system embodiment, wherein the isolated total nucleic acid is fragmented to fragments having a size ranging from 2.4-3.2 kilobase pairs (kb) prior to contacting the isolated total nucleic acid with the methyl binding protein.

Illustrative embodiment 34 is the system of any preceding or subsequent illustrative system embodiment, wherein the methyl binding protein is a bifunctional polypeptide comprising: (i) an Fc portion of an antibody; (ii) a short flexible peptide linker; and (iii) a DNA-binding domain of an MBD2 protein.

Illustrative embodiment 35 is the system of any preceding or subsequent illustrative system embodiment, wherein the size of the FMRI amplification product in the fraction enriched for methylated nucleic acid and/or the fraction enriched for unmethylated nucleic acid and/or the total nucleic acid is measured by capillary electrophoresis.

Illustrative embodiment 36 is the system of any preceding or subsequent illustrative system embodiment, further comprising a component and/or station for determining whether the subject has either: (i) partial methylation of FMRI; (ii) both a methylated and unmethylated copy of FMRI, or (iii) full methylation of FMRI.

Illustrative embodiment 37 is the system of any preceding or subsequent illustrative system embodiment, further comprising a component and/or station for determining whether the subject has either: (i) partial methylation of an expanded FMRI allele; (ii) both a methylated and unmethylated expanded FMRI allele; or (iii) full methylation of an expanded FMRI allele.

Illustrative embodiment 38 is the system of any preceding or subsequent illustrative system embodiment, further comprising a data processor and/or a non-transitory computer readable storage medium containing instructions which, when executed on the one or more data processors, cause the one or more data processors to perform actions programmable software to control any of the stations and/or components of the system.

Illustrative embodiment 39 is the system of any preceding or subsequent illustrative system embodiment, further comprising a non-transitory computer readable storage medium containing instructions for analyzing the data from FRAX PCR and/or FRAX me-PCR. Illustrative embodiment 40 is the system of any preceding or subsequent illustrative system embodiment, further comprising a non-transitory computer readable storage medium containing instructions to perform the steps of providing the size and abundance of each PCR amplicon from FRAX PCR (total nucleic acid) and FRAX mePCR as peak size and height.

Illustrative embodiment 41 is the system of any preceding or subsequent illustrative system embodiment, wherein the analysis checks the sizing standards and peak heights to ensure that the GS-PCR CE data quality are acceptable for analysis.

Illustrative embodiment 42 is the system of any preceding or subsequent illustrative system embodiment, wherein the sample's GS-PCR peak data derived from total DNA is used to calculate the CGG repeat number for each allele.

Illustrative embodiment 43 is the system of any preceding or subsequent illustrative system embodiment, wherein using these CGG repeat numbers, the GS-PCR CE data of the methylated and unmethylated DNA fractions in the HEX and FAM channels, respectively, is scanned to identify corresponding peaks that are above the allele-specific peak height thresholds.

Illustrative embodiment 44 is the system of any preceding or subsequent illustrative system embodiment, wherein besides the peaks detected using total DNA, the methylation status calling algorithm will not identify additional peaks in the methylated or unmethylated fraction.

Illustrative embodiment 45 is the system of any preceding or subsequent illustrative system embodiment, wherein optionally, detection of the same peak in total DNA as well as in either the methylated or unmethylated fraction, but not both, indicates fully methylated or unmethylated status, respectively, for the allele and/or if the same peak occurs in both fractions as well as total DNA, the allele is called as partially methylated.

Illustrated embodiment 46 is a computer-program product tangibly embodied in a non-transitory machine-readable storage medium, including instructions configured to perform any of the preceding or subsequent method embodiments or use any of the compositions of any of the preceding or subsequent composition embodiments or use any of the kits of any of the preceding or subsequent kit embodiments or to run any of the components and/or stations of any of the preceding or subsequent composition embodiments.

Illustrated embodiment 47 is a computer-program product of any preceding or subsequent illustrative computer-program product embodiment, comprising the steps of:

determining the presence or absence of an FMRI amplification product in both a fraction of nucleic acid enriched for methylated nucleic acid and a fraction enriched for unmethylated nucleic acid;

determining the size of the FMRI amplification product in at least one of the fraction enriched for methylated nucleic acid and the fraction enriched for unmethylated nucleic acid;

comparing the size of the amplification product for the fraction enriched for methylated nucleic acid and the fraction enriched for unmethylated nucleic acid to the size of the at least one

FMRI amplification product for total nucleic acid; and determining size of the FMRI amplification product in at least one of the fraction enriched for methylated nucleic acid and the fraction enriched for unmethylated nucleic acid as being the size of the at least one FMRI amplification product for the total nucleic acid.

Illustrated embodiment 48 is a computer-program product of any preceding or subsequent illustrative computer-program product embodiment, comprising instructions configured to cause one or more data processors to perform actions to direct at least one of the steps of:

isolating total nucleic acid from the sample, the total nucleic acid comprising both methylated nucleic acid and unmethylated nucleic acid;

contacting a first aliquot of the isolated total nucleic acid with a methyl binding protein;

isolating a portion of the unbound nucleic acid, thereby generating a nucleic acid fraction enriched for unmethylated nucleic acid;

isolating the bound nucleic acid, thereby generating a nucleic acid fraction enriched for methylated nucleic acid; conducting PCR amplification of the FMRI gene using an aliquot of nucleic acid from the fraction enriched for methylated nucleic acid;

conducting PCR amplification of the FMRI gene using an aliquot of nucleic acid from the fraction enriched for unmethylated nucleic acid; and determining the presence or absence of an FMRI amplification product in both the fraction enriched for methylated nucleic acid and the fraction enriched for unmethylated nucleic acid.

Illustrative embodiment 49 is the computer-program product of any preceding or subsequent illustrative computer-program product embodiment, further comprising a non-transitory computer readable storage medium containing instructions for analyzing the data from FRAX PCR and/or FRAX me-PCR.

Illustrative embodiment 50 is the computer-program product of any preceding or subsequent illustrative computer-program product embodiment, further comprising a non-transitory computer readable storage medium containing instructions to perform the steps of providing the size and abundance of each PCR amplicon from FRAX PCR (total nucleic acid) and FRAX mePCR as peak size and height.

Illustrative embodiment 51 is the computer-program product of any preceding or subsequent illustrative computer-program product embodiment, wherein the analysis checks the sizing standards and peak heights to ensure that the GS-PCR CE data quality are acceptable for analysis.

Illustrative embodiment 52 is the computer-program product of any preceding or subsequent illustrative computer-program product embodiment, wherein the sample's GS-PCR peak data derived from total DNA is used to calculate the CGG repeat number for each allele.

Illustrative embodiment 53 is the computer-program product of any preceding or subsequent illustrative computer-program product embodiment, wherein using these CGG repeat numbers, the GS-PCR CE data of the methylated and unmethylated DNA fractions in the HEX and FAM channels, respectively, is scanned to identify corresponding peaks that are above the allele-specific peak height thresholds.

Illustrative embodiment 54 is the computer-program product of any preceding or subsequent illustrative computer-program product embodiment, wherein besides peaks detected using total DNA, the methylation status calling algorithm will not identify additional peaks in the methylated or unmethylated fraction.

Illustrative embodiment 55 is the computer-program product of any preceding or subsequent illustrative computer-program product embodiment, wherein optionally, detection of the same peak in total DNA as well as in either the methylated or unmethylated fraction, but not both, indicates fully methylated or unmethylated status, respectively, for the allele and/or if the same peak occurs in both fractions as well as total DNA, the allele is called as partially methylated.

Illustrative embodiment 56 is the method of any preceding or subsequent illustrative method embodiment, wherein the analysis checks the sizing standards and peak heights to ensure that the GS-PCR CE data quality are acceptable for analysis.

Illustrative embodiment 57 is the method of any preceding or subsequent illustrative method embodiment, wherein the sample's GS-PCR peak data derived from total DNA is used to calculate the CGG repeat number for each allele.

Illustrative embodiment 58 is the method of any preceding or subsequent illustrative method embodiment, wherein using these CGG repeat numbers, the GS-PCR CE data of the methylated and unmethylated DNA fractions in the HEX and FAM channels, respectively, is scanned to identify corresponding peaks that are above the allele-specific peak height thresholds.

Illustrative embodiment 59 is the method of any preceding or subsequent illustrative method embodiment, wherein besides the peaks detected using total DNA, the methylation status calling algorithm will not identify additional peaks in the methylated or unmethylated fraction.

Illustrative embodiment 60 is the method of any preceding or subsequent illustrative method embodiment, wherein optionally, detection of the same peak in total DNA as well as in either the methylated or unmethylated fraction, but not both, indicates fully methylated or unmethylated status, respectively, for the allele and/or if the same peak occurs in both fractions as well as total DNA, the allele is called as partially methylated.

That which is claimed is:

1. A method for determining methylation of an FMRI gene in a sample from a subject comprising the steps of:
isolating total nucleic acid from the sample, the total nucleic acid comprising both methylated nucleic acid and unmethylated nucleic acid;
contacting a first aliquot of the isolated total nucleic acid with a methyl binding protein;
isolating a portion of the unbound nucleic acid, thereby generating a nucleic acid fraction enriched for unmethylated nucleic acid;
isolating the bound nucleic acid, thereby generating a nucleic acid fraction enriched for methylated nucleic acid;
conducting PCR amplification of the FMRI gene using an aliquot of nucleic acid from the fraction enriched for methylated nucleic acid;
conducting PCR amplification of the FMRI gene using an aliquot of nucleic acid from the fraction enriched for unmethylated nucleic acid; and
determining the presence or absence of an FMRI amplification product in both the fraction enriched for methylated nucleic acid and the fraction enriched for unmethylated nucleic acid.

2. The method of claim 1, further comprising determining the size of the FMRI amplification product in at least one of the fraction enriched for methylated nucleic acid and the fraction enriched for unmethylated nucleic acid.

3. The method of claim 2, further comprising determining the number of CGG repeats at the FMRI gene for the amplification product in at least one of the fraction enriched for methylated nucleic acid and the fraction enriched for unmethylated nucleic acid.

4. The method of claim 1, further comprising performing PCR amplification of the FMRI gene using a second aliquot of nucleic acid from the total nucleic acid and determining the size of at least one FMRI amplification product.

5. The method of claim 4, further comprising determining the number of CGG repeats at the FMRI gene for the at least one amplification product obtained from the total nucleic acid fraction.

6. The method of claim 2, wherein determining the size of the FMRI amplification product in at least one of the fraction enriched for methylated nucleic acid and the fraction enriched for unmethylated nucleic acid comprises comparing the size of the amplification products for the fraction enriched for methylated nucleic acid and/or the fraction enriched for unmethylated nucleic acid to the size of the at least one FMRI amplification product for total nucleic acid.

7. The method of claim 6, wherein the size of the FMRI amplification product in at least one of the fraction enriched for methylated nucleic acid and the fraction enriched for unmethylated nucleic acid is determined to be the size of the at least one FMRI amplification product for total nucleic acid.

8. The method of claim 1, wherein at least one primer used to generate an FMRI amplification product is labeled with a detectable moiety.

9. The method of claim 8, wherein the at least one primer used to generate an amplification product from the FMRI gene from the fraction enriched for methylated nucleic acid and/or the fraction enriched for unmethylated nucleic acid and/or the total nucleic acid have the same sequence but are labeled with a different detectable moiety.

10. The method of claim 8, wherein the detectable moiety is a fluorescent dye.

11. The method of claim 10, wherein the fluorescent dye used for detection of the amplification products from the fraction enriched for unmethylated nucleic acid and/or the total nucleic acid is 6-carboxyfluorescein (FAM) and the dye used for detection of the amplification product from the fraction enriched for methylated nucleic acid is hexachlorofluorescein (HEX).

12. The method of claim 1, wherein the isolated total nucleic acid is fragmented to fragments having a size ranging from 2.4-3.2 kilobase pairs (kb) prior to contacting the isolated total nucleic acid with the methyl binding protein.

13. The method of claim 1, wherein the methyl binding protein is a bifunctional polypeptide comprising: (i) an Fc portion of an antibody; (ii) a short flexible peptide linker; and (iii) a DNA-binding domain of an MBD2 protein.

14. The method of claim 1, wherein the size of the FMRI amplification product in the fraction enriched for methylated nucleic acid and the fraction enriched for unmethylated nucleic acid is measured by capillary electrophoresis.

15. The method of claim 1, further comprising determining whether the subject has either: (i) partial methylation of FMRI; (ii) both a methylated and unmethylated copy of FMRI, or (iii) full methylation of FMRI and/or

16. The method of any of claim 15, further comprising determining whether the subject has either: (i) partial methylation of an expanded FMRI allele; (ii) both a methylated and unmethylated expanded FMRI allele; or (iii) full methylation of an expanded FMRI allele.

17. A composition comprising at least one primer having the sequence of SEQ ID NOs: 1-3.

18. A system for determining methylation of an FMRI gene in a sample from a subject comprising:
a component for isolating total nucleic acid from the sample;
a component for fragmenting the isolated total nucleic acid to a specific size range;
a component for contacting a first portion of the fragmented nucleic acid with a methyl binding protein
a component for collecting a fraction of the nucleic acid that is enriched for methylated nucleic acid and a fraction of the nucleic acid that is enriched for unmethylated nucleic acid;
a component to perform PCR amplification of the FMRI gene separately for the nucleic acid that is enriched for methylated nucleic acid and the fraction of the nucleic acid that is enriched for unmethylated nucleic acid; and
a component to determine if FMRI gene sequences are present in the nucleic acid fraction enriched for methylated nucleic acid and/or the nucleic acid fraction enriched for unmethylated nucleic acid or both.

19. A computer readable media comprising instructions to perform a method for determining methylation of an FMRI gene in a sample from a subject comprising the steps of:
determining the presence or absence of an FMRI amplification product in both a fraction of nucleic acid enriched for methylated nucleic acid and a fraction enriched for unmethylated nucleic acid;
determining the size of the FMRI amplification product in at least one of the fraction enriched for methylated nucleic acid and the fraction enriched for unmethylated nucleic acid;
comparing the size of the amplification product for the fraction enriched for methylated nucleic acid and the fraction enriched for unmethylated nucleic acid to the size of the at least one FMRI amplification product for total nucleic acid; and
determining size of the FMRI amplification product in at least one of the fraction enriched for methylated nucleic acid and the fraction enriched for unmethylated nucleic acid as being the size of the size of the at least one FMRI amplification product for total nucleic acid.


SEQUENCE LISTING (4 sequences)

SEQ ID NO: 1 - DNA, 30 bp, synthetic construct
  source 1..30: mol_type = other DNA
gctcagctccgtttcggtttcacttccggt

SEQ ID NO: 2 - DNA, 30 bp, synthetic construct
  source 1..30: mol_type = other DNA
agccccgcacttccaccaccagctcctcca

SEQ ID NO: 3 - DNA, 30 bp, synthetic construct; identical to SEQ ID NO: 2
  source 1..30: mol_type = other DNA

SEQ ID NO: 4 - DNA, 30 bp, synthetic construct; identical to SEQ ID NO: 2
  source 1..30: mol_type = other DNA